(19) United States
(12) Patent Application Publication
(10) Pub. No.: US 2003/0105279 A1
(43) Pub. Date: Jun. 5, 2003

(54) Aromatic and heteroaromatic acid halides for synthesizing polyamides

(75) Inventor: Dennis P. Phillion, St. Charles, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/232,244

(22) Filed: Aug. 30, 2002

Related U.S. Application Data
(63) Continuation-in-part of application No. 10/061,617, filed on Feb. 1, 2002.
(60) Provisional application No. 60/316,151, filed on Aug. 30, 2001.

(51) Int. Cl. C07K 7/08; C07D 285/12; C07D 285/14; C07D 277/18; C07D 263/16; C07K 7/06

(52) U.S. Cl. 530/324; 548/136; 548/194; 548/233; 548/328.1

(57) ABSTRACT

The present invention is directed to protected amino acid halide monomers and oligomers, and to their use in the efficient sythesis of polyamides. The present invention is further directed to the use of α-haloenamine reagents, which may optionally be immobilized, for the preparation of the amino acid halides.

### REFERENCE TO RELATED APPLICATIONS

This application claims priority from U.S. Provisional Application Serial No. 60/316,151, filed on Aug. 30, 2001 and U.S. application Ser. No. 10/061,617, filed on Feb. 1, 2002, both of which are incorporated in their entireties herein by reference.

### BACKGROUND OF THE INVENTION

The present invention relates in general to the preparation of acid halides and their use in the synthesis of polyamides. More particularly, the present invention related to the preparation of aromatic and heteroaromatic acid chlorides and bromides using α-chloroenamines and α-bromoenamines, respectively, which may optionally be immobilized on a support. The present invention is further related to the use of these aromatic and heteroaromatic acid halides in the synthesis of polyamides.

The regulation of gene expression is mediated by repressor, activator and enhancer proteins that control expression by site-specific binding to nucleic acids such as DNA. Small molecules that selectively control or disrupt such regulatory binding processes are potentially useful as agents for the control of gene expression. Sequence-specific DNA-binding compounds are therefore highly sought as regulators of gene expression, for use in biotechnology as agricultural agents and as gene-specific drugs. Such sequence-specific DNA-binding compounds include, for example, triplex-forming oligonucleotides, peptide nucleic acids, and polyamides that bind to the minor groove of DNA.

In particular, the stability and simplicity of structure of minor groove-binding polyamides make such molecules especially suitable for most sequence-specific DNA-binding applications. For example, polyamides containing N-methylimidazole and N-methylpyrrole amino acids are especially promising as DNA-binding compounds because they bind to form antiparallel, side-by-side dimeric complexes with the minor groove of DNA, and because the sequence specificity of the binding can be controlled by changing the linear sequence of imidazole and pyrrole amino acids. (See, e.g., Wade et al., J. Am. Chem. Soc., 114: 8783, 1992.) Modified polyamide molecules that include a simple “hairpin motif” to link polyamide subunits have been described which show increased DNA binding affinity and specificity over individual unlinked polyamide pairs. (See, e.g., Mrksich et al., J. Am. Chem. Soc., 116: 7983, 1994.)

In general, a useful approach toward further development and testing of novel binding agents, including polyamides, includes the use of combinatorial chemistry to develop libraries of molecules that are then analyzed using high throughput screening techniques to identify DNA sequence selectivities and binding affinities. However, the discovery and analysis of useful polyamides have been slowed by the technical difficulties associated with designing and synthesizing them. In particular, heteroaromatic amines are unstable and formation of the amide bonds require long coupling times which makes synthesis of libraries difficult.

A method has been described for solid phase synthesis of certain polyamide compounds containing imidazole and pyrrole carboxamides (see, e.g., U.S. Pat. No. 6,090,947, which is incorporated herein by reference). This method advantageously utilizes t-butoxycarbonyl (“BOC”)-protected heterocyclic amino acids as monomeric building blocks in automated solid-phase synthesis. Highly activating coupling agents, such as carbodiimides and HBTU (O-benzotriazol-1-yl-N,N,N′,N′-tetramethyluronium hexafluorophosphate), are used to form the amide linkages, contributing to the growing polymer chain. Specifically, 1-methyl-4-(BOC amino)pyrrole-2-carboxylic acid, 1-methyl-3-methoxy-4-(BOC amino)pyrrole-2-carboxylic acid, and 1-methyl-4-(BOC amino)imidazole-2-carboxylic acid are described as being useful monomeric building blocks.

However, although peptide coupling agents, such as carbodiimides and HBTU, are known to form the amide linkages in the growing polymer chain, the imidazole-CONH-imidazole and pyrrole-CONH-imidazole amide linkages have not been easily formed in high yields. Furthermore, the reactions using such agents typically produce a mixture of products, at least in part due to the fact that a large excess of such agents are needed in order for the reaction to proceed, thus requiring laborious processing to obtain the desired end-products in sufficient purity. Ultimately, the reactions only produce the polyamides in very small quantities. Therefore, such methods have limited application for preparing large numbers of pure polyamides (libraries) for high throughput screening.

Alternatively, certain acid chloride compounds have been used to prepare certain polyamides and related structures. For example, oxalyl chloride and thionyl chloride can be reacted with certain carboxylic acids to prepare acid chlorides that can then be used to prepare polyamides. (See, e.g., Krowicki et al., J. Org. Chem., 52 (16): 3493-501, 1987 and Konig et al., Chem. Commun., 1998, 605-6.) However, such methods are of limited value for a number of reasons. For example, the acid chlorides are not always produced at high levels of purity, and the methods involve harsh chemistry, such as elevated temperature or acidic conditions, which can lead to reactions with the protecting groups or heterocyclic rings. In addition, two steps are required to generate the acid chloride from the carboxylic acid. Finally, because of the manner by which the amino groups are protected, additional steps are required to remove the protecting group, steps which are not well-suited for automated, solid phase polyamide synthesis.

As a result, the known acid chloride compounds and related methods of preparing or using such compounds have limited utility for synthesizing a wide variety of polyamides, particularly heterocyclic polyamides, in high yield. A need therefore remains for reagents and methods that provide for the efficient preparation of polyamides in variety and quantity.

### SUMMARY OF THE INVENTION

Among the features of the present invention, therefore, is the provision of an improved process for preparing polyamides which are especially useful in binding to the minor groove of DNA; the provision of such a process which may be carried out in solution phase or solid phase; the provision of such a process wherein a low equivalency ratio of monomer to growing polymer chain end may be used; and, the provision of such a process wherein novel amino acid halides are used as monomeric or oligomeric building blocks.

Further among the features of the present invention is the provision of an amino acid halide, and an efficient process for the preparation thereof, which is particularly well-suited for use in the preparation of polyamides; the provision of such an amino acid halide having improved purity; the provision of a process for preparing such an amino acid halides in high yield; the provision of such an amino acid halide which enables the preparation of polyamides having high purity and in high yield; the provision of a process for preparing such amino acid halides wherein a novel α-haloenamine is employed; and, the provision of such an α-haloenamine which may optionally be immobilized on a support, the resulting α-haloenamine being particularly useful in high-throughput, automated and other systems where ease of separation is desired.

Briefly, therefore, the present invention is directed to an amino acid halide comprising a 5-member heteroaromatic ring, the amino acid halide having a formula (4), (5) or (6):

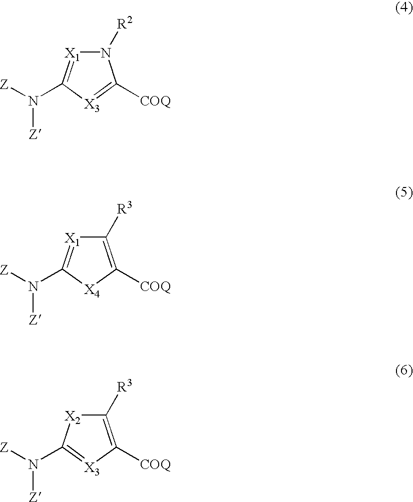

wherein:

Q is Cl or Br;

Z and Z′ are independently hydrogen or an amine protecting group which may be the same or different, provided that at least one of Z and Z′, together with the nitrogen atom to which they are attached, form a carbamate moiety;

X₁ is N or CR¹;

X₂ is O, S or NR¹;

X₃ is N or CR⁴;

X₄ is O or S;

R¹ is independently selected from hydrogen and substituted or unsubstituted alkyl;

R² is independently selected from hydrogen, substituted or unsubstituted C₁-C₁₀ alkyl, substituted or unsubstituted C₂-C₁₀ alkenyl, or substituted or unsubstituted C₂-C₁₀ alkynyl, provided that when X₃ is CH and X₁ is N, R² is not methyl;

R³ is independently selected from hydrogen, substituted or unsubstituted C₁-C₁₀ alkyl, substituted or unsubstituted C₂-C₁₀ alkenyl, substituted or unsubstituted C₂-C₁₀ alkynyl or halo, provided that (i) when X₂ is S and X₃ is N, R³ is not hydrogen, and (ii) when X₁ is N and X₄ is S, R³ is not hydrogen or methyl; and,

R⁴ is independently selected from hydrogen, hydroxy or alkoxy.

The present invention is further directed to an amino acid halide oligomer comprising a 5-member heteroaromatic or a 6-member aromatic or heteroaromatic ring, the oligomer having a formula (10), (11) or (12):

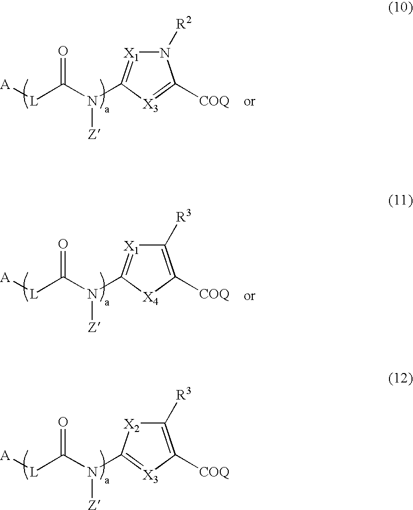

wherein:

Q is Cl or Br;

a is at least about 1 and represents the number of tandem units present in the oligomer;

A is H or NZZ′;

Z and Z′ are independently hydrogen or an amine protecting group which is the same or different, provided that at least one of Z and Z′, together with the nitrogen atom to which they are attached, form a carbamate moiety;

X₁ is N or CR¹;

X₂ is O, S, NR¹, —CR¹═CR¹′—, —CR¹═N—, —N═CR¹— or —N═N—;

X₃ is N or CR⁴;

X₄ is O or S;

R¹ and R¹′ are independently selected from hydrogen and substituted or unsubstituted alkyl;

R² is independently selected from hydrogen, substituted or unsubstituted C₁-C₁₀ alkyl, substituted or unsubstituted C₂-C₁₀ alkenyl, or substituted or unsubstituted C₂-C₁₀ alkynyl;

R³ is independently selected from hydrogen, substituted or unsubstituted C₁-C₁₀ alkyl, substituted or unsubstituted C₂-C₁₀ alkenyl, substituted or unsubstituted C₂-C₁₀ alkynyl or halo;

R⁴ is independently selected from hydrogen, hydroxy or alkoxy; and,

L is a subunit of the tandem unit independently selected from substituted or unsubstituted alkylene, substituted or unsubstituted arylene and substituted or unsubstituted heteroarylene, wherein (i) L may be the same or different for each tandem unit, and (ii) no more than about one L is present which enables a hairpin turn therein.

The present invention is still further directed to a process for preparing the amino acid halide monomers noted above. The process comprises contacting an α-haloenamine and an amino-protected, 5-member heteroaromatic carboxylic acid, wherein said carboxylic acid corresponds to formula (4), (5) or (6), except that Q is OH and not Cl or Br.

The present invention is still further directed to a process for preparing the amino acid halide oligomers noted above. The process comprises contacting an α-haloenamine and an amino-protected, 5-member heteroaromatic or 6-member aromatic or heteroaromatic carboxylic acid, wherein said carboxylic acid corresponds to formula (10), (11) or (12), except that Q is OH and not Cl or Br.

The present invention is still further directed to a process for preparing an amino acid halide, P—COQ, wherein P represents a substituted or unsubstituted, 5-member heteroaromatic or 6-member aromatic or heteroaromatic ring which has a protected amino group bound thereto, CO represents a carbonyl group bound to the ring, and Q represents a halo group bound to the carbonyl carbon. The process comprising contacting an α-haloenamine, which may or may not be immobilized, and a carboxylic acid, P—CO₂H, that corresponds structurally to the amino acid halide except that Q is hydroxy instead of halo.

In one particular emobidment of these processes, the α-haloenamine has the formula:

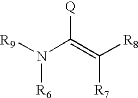

wherein:

R₆ and R₉ are independently hydrocarbyl, substituted hydrocarbyl, hydrocarbyloxy or substituted hydrocarbyloxy;

R₇ and R₈ are independently hydrogen, hydrocarbyl, substituted hydrocarbyl, hydrocarbylthio, substituted hydrocarbylthio, hydrocarbylcarbonyl, substituted hydrocarbylcarbonyl, hydrocarbyloxycarbonyl, substituted hydrocarbyloxycarbonyl, phosphinyl, thiophosphinyl, sulfinyl, sulfonyl, halo, cyano, or nitro; and

Q is chloro or bromo;

provided that, when the α-haloenamine is immobilized, at least one of R₆, R₇, R₈ and R₉ comprises a support which enables physical separation of the reagent from a liquid mixture.

The present invention is further directed to processes for employing such amino acid halides to prepare polyamides. More specifically, the present invention is directed to a process for preparing a polyamide oligomer or polymer, the process comprising: (a) forming a population of amino-protected, amino acid halide building block units comprising (i) a 5-member heteroaromatic ring and having a formula (4), (5) or (6), or (ii) a 5-member heteroaromatic or 6-member aromatic or heteroaromatic ring and having formula (10), (11) or (12), as described above; (b) combining a member of the population of amino-protected, amino acid halide building block units with a moiety comprising an unprotected amino functionality to form a reaction product possessing an amide linkage and a protected amino functionality; (c) deprotecting the amino group of the reaction product; and, (d) repeating steps (b) and (c) at least once, wherein the deprotected reaction product formed in step (c) is used as the moiety comprising an unprotected amino functionality in a subsequent step (b).

The reagents, compounds and methods of the present invention are particularly useful for generating polyamide libraries for systematic testing of polyamide compounds as modulators of gene transcription.

### DETAILED DESCRIPTION OF THE PREFERRED EMBODIMENTS

Generally speaking, in one embodiment, the present invention is directed to a method of preparing amino acid halides, in particular a method using an α-haloenamine which may or may not be immobilized on a support, as well as to various amino acid halides resulting therefrom. In another embodiment, the present invention is directed to methods of using such amino acid halides to prepare polyamides. Experience to-date suggests these amino acid halides are particularly well-suited for use as building blocks in the preparation of polyamides, especially in automated, solid phase preparations methods, at least in part because of their improved stability and the ease with which the protecting groups utilized herein (e.g., groups which form carbamates with the nitrogen atom of the amino group) can be attached and removed. The use of such acid halides enables the more efficient preparation of polyamides; for example, the ratio of monomer to growing polymer chain end is significantly lower than conventional methods (wherein the ratio of monomer to growing polymer is typically 4:1).

More specifically, as further described herein below, in one embodiment the present invention is directed to a method of preparing an amino acid halide monomer or oligomer, the amino acid halide comprising a 5-member heteroaromatic or 6-member aromatic or heteroaromatic ring and having a formula (1), (2) or (3):

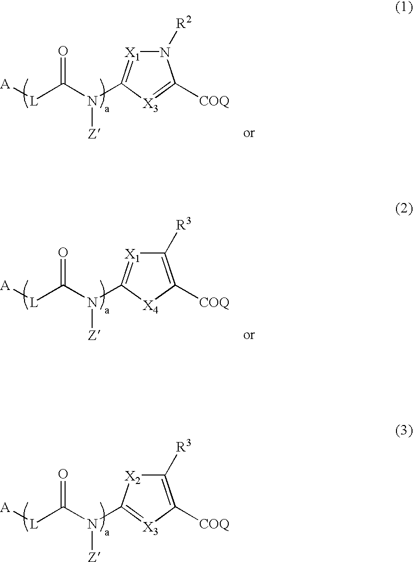

wherein:

Q is halo (e.g., chloro or bromo);

a is greater than or equal to 0 and, when greater than 0, represents the number of tandem units present in an oligomeric amino acid halide;

A is H or NZZ′, provided that when a is 0, A is NZZ′;

Z and Z′ are independently hydrogen or an amine protecting group, which may be the same or different, provided at least one is a protecting group, such that each N present is protected thereby (or protected in some other manner, for puposes of the reactions described herein below);

X₁ is N or CR¹;

X₂ is O, S, NR¹, —CR¹═CR¹′—, —CR¹═N—, —N═CR¹— or —N═N—;

X₃ is N or CR⁴;

X₄ is O or S;

R¹ and R¹′ are independently selected from hydrogen and substituted or unsubstituted alkyl (e.g., methyl, ethyl, propyl, butyl, pentyl, etc.);

R² is independently selected from hydrogen, substituted or unsubstituted alkyl (e.g., C₁ or C₂ to C₁₀ alkyl), substituted or unsubstituted alkenyl (e.g., C₂-C₁₀ alkenyl), or substituted or unsubstituted alkynyl (e.g., C₂-C₁₀ alkynyl);

R³ is independently selected from hydrogen, substituted or unsubstituted alkyl (e.g., C₁ or C₂ to C₁₀ alkyl), substituted or unsubstituted alkenyl (e.g., C₂-C₁₀ alkenyl), substituted or unsubstituted alkynyl (e.g., C₂-C₁₀ alkynyl) or halo (e.g., chloro, bromo, fluoro, etc.);

R⁴ is independently selected from hydrogen, hydroxy or alkoxy (e.g., substituted or unsubstituted methoxy, ethoxy, propoxy, butoxy, pentoxy, etc.); and,

L is a subunit of the tandem unit independently selected from substituted or unsubstituted alkylene, arylene and heteroarylene, wherein (i) L may be the same or different for each tandem unit present, and (ii) no more than about one L is present which enables a hairpin (e.g., beta or gamma) turn therein.

In general, the protecting group may be essentially any group known in the art which will prevent the amino substituent from reacting with the acid halide substituent of another amino acid halide molecule (e.g., a group which prevent the protected amino acid halide from reacting with itself), or a reagent used to generate the acid halide functionality (e.g., an α-haloenamine, as further described herein). Suitable protecting groups are identified, for example, in Protective Groups in Organic Synthesis by T. W. Greene and P. G. M. Wuts, John Wiley and Sons, 3rd ed. 1999, the entire contents of which is incorporated herein by reference. In addition, the protecting group is preferably selected so as to increase the solubility of the molecule of which it is a part in a desired reaction medium or mixture. For example, as further described herein, one approach for preparing the protected amino acid halides of the present invention is to react a corresponding amino protected carboxylic acid or thiocarboxylic acid with an α-haloenamine, the protecting group preferably being selected so as to increase the solubility of the carboxylic acid in the reaction medium or mixture.

Particularly preferred protecting groups for use in the present invention include those which are selectively removable under (i) acidic conditions (e.g., t-butoxycarbonyl or “Boc”), (ii) basic or mildly basic conditions (e.g., 9-fluoroenylmethoxy carbonyl or “Fmoc”), (iii) non-reducing conditions, (iv) reducing conditions, or various combinations thereof (e.g., acid, basic or reducing; acid, basic or non-reducing; etc.). Such protecting groups include those which, together with the amino group nitrogen to which they are attached, form a carbamate moiety. Boc and Fmoc are exemplary and are particularly preferred because they are well-suited for automated, solid phase synthesis of polyamides.

In this regard it is to be noted that, as used herein, “mildly basic” generally refers to techniques for removing the protecting group which are less aggressive than, for example, a saponification; that is, “mildly basic” refers to techniques for removing the protecting group which utilize a secondary amine (or similar reagent in terms of reactivity toward the protecting group), rather than a hydroxide (or similar reagent in terms of reactivity toward the protecting group).

In this regard it is to be further noted, however, that the protecting group(s) utilized may be other than herein described without departing from the scope of the present invention. In addition, it is to be noted that, as indicated, in some embodiments one protecting group is used, Z′ thus being hydrogen, while in other embodiments two protecting groups are used. In such instances, the two protecting groups may be the same or different. Furthermore, in yet other embodiments (e.g., when a is 1 or more), both A and Z′ may be hydrogen, the nitrogen atom present in each tandem unit therefore being protected by the amide linkage of which it is a part.

Finally, It is to be noted that, in some preferred embodiments, the halogen present is selected from chloro or bromo (i.e., in at least some embodiments the present invention is preferably directed to amino acid chlorides and bromides), and more preferably is chloro, and, as appropriate (i.e., in view of the limitations described elsewhere herein), (i) R² and R³ are hydrogen or methyl, (ii) X₃ is N or CH, (iii) X₁ is N or CH, and (iv) X₄ is S or O.

I. Protected Amino Acid Halides

A. Monomers

In one embodiment, the present invention is directed to a protected amino acid halide monomer comprising a 5-member heteroaromatic ring, the protected amino and acid halide groups or substituents preferably having a 1,3 positional relationship on the ring (i.e., the substituents being separated by 1 carbon or heteroatom in the ring). Without being held to any particular theory, this spacing of the substituents on this ring is believed to enable the polyamides derived therefrom to more efficiently bind within the minor groove of DNA.

Accordingly, the monomeric amino acid halides of the present invention preferably having a formula (4), (5) or (6):

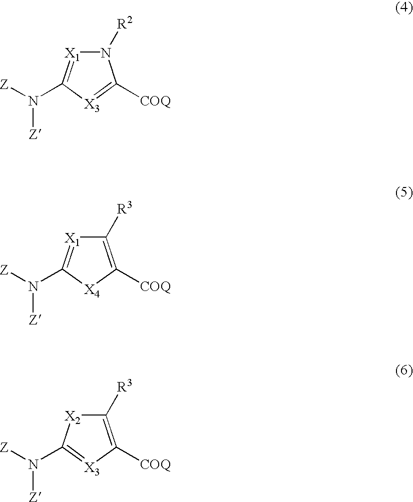

wherein:

Q is Cl or Br;

Z and Z′ are independently hydrogen or an amine protecting group which may be the same or different, provided that at least one of Z and Z′, together with the nitrogen atom to which they are attached, form a carbamate moiety;

X₁ is N or CR¹;

X₂ is O, S or NR¹;

X₃ is N or CR⁴;

X₄ is O or S;

R¹ is independently selected from hydrogen and substituted or unsubstituted alkyl;

R² is independently selected from hydrogen, substituted or unsubstituted C₁-C₁₀ alkyl, substituted or unsubstituted C₂-C₁₀ alkenyl, or substituted or unsubstituted C₂-C₁₀ alkynyl, provided that when X₃ is CH and X₁ is N, R² is not methyl (i.e., R² is in this instance hydrogen, substituted or unsubstituted C₂-C₁₀ alkyl, substituted or unsubstituted alkenyl or substituted or unsubstituted alkynyl, as described herein);

R³ is independently selected from hydrogen, substituted or unsubstituted C₁-C₁₀ alkyl, substituted or unsubstituted C₂-C₁₀ alkenyl, substituted or unsubstituted C₂-C₁₀ alkynyl or halo, provided that (i) when X₂ is S and X₃ is N, R³ is not hydrogen (i.e., R³ is in this instance substituted or unsubstituted alkyl, substituted or unsubstituted alkenyl or substituted or unsubstituted alkynyl, as described herein), and (ii) when X₁ is N and X₄ is S, R³ is not hydrogen or methyl (i.e., R³ is in this instance substituted or unsubstituted C₂-C₁₀ alkyl, substituted or unsubstituted alkenyl or substituted or unsubstituted alkynyl, as described herein); and,

R⁴ is independently selected from hydrogen, hydroxy or alkoxy.

Among the preferred 5-member, heteroaromatic ring structures from which the amino acid halides of the present invention are derived include those selected from, for example, substituted or unsubstituted pyrrole, substituted or unsubstituted imidazole, substituted or unsubstituted furan, substituted or unsubstituted pyrazole, substituted or unsubstituted thiophene, substituted or unsubstituted oxazole, substituted or unsubstituted thiazole and substituted or unsubstituted 1,2,4-triazole. However, without being held to a particular theory, it is believed that binding in the minor groove of DNA is aided by the presence of a nitrogen atom at the one position of the ring; that is, it is believed preferable to have a nitrogen atom present between the acid halide and amino substituents.

Accordingly, in some particularly preferred embodiments of the monomer, X₃ is N. Such monomers therefore have a formula (7A) or (9A):

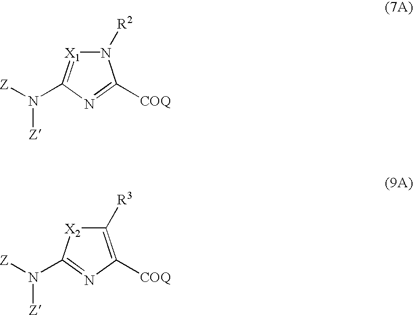

wherein Q, Z, Z′, X₁, X₂, R¹, R² and R³ are as defined above with respect to formulas (4) and (6), respectively. Exemplary embodiments include those having the formula:

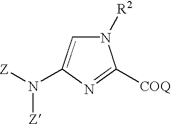

wherein:

Z′ is preferably hydrogen;

Z is Boc or Fmoc, prefererably Boc;

R² is preferably hydrogen or methyl; and,

Q is chloro or bromo, preferably chloro.

In an alternative embodiment, however, X₃ is CR₄, the monomer having a formula (7B) or (9B):

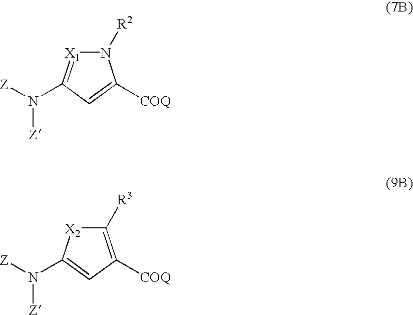

wherein Q, Z, Z′, X₁, X₂, R¹, R² and R³ are as defined above with respect to formulas (4) and (6), respectively. Exemplary embodiments include those having the formula:

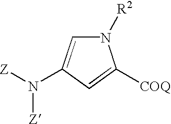

wherein:

Z′ is preferably hydrogen;

Z is Boc or Fmoc, prefererably Boc;

R² is preferably hydrogen or methyl; and,

Q is chloro or bromo, preferably chloro.

Among the additional, preferred embodiments are: for formula (4), those wherein Z′ is H; Z is Boc or Fmoc, and more preferably is Boc; Q is chloro or bromo, and more preferably is chloro; X₁ is N; X₃ is CH or CCH₃; and, R² is H or CH₃; for formula (5), those wherein Z′ is H; Z is Boc or Fmoc, and more preferably is Boc; Q is chloro or bromo, and more preferably is chloro; X₁ is N; X₄ is O; and, R³ is H or CH₃; and, for formula (6), those wherein Z′ is H; Z is Boc or Fmoc, and more preferably is Boc; Q is chloro or bromo, and more preferably is chloro; X₂ is NH; X₃ is CH or N; and, R³ is H or CH₃.

B. Oligomers

In another preferred embodiment, the present invention is directed to an oligomeric amino acid halide, which can be prepared and utilized by the methods described herein. The oligomer comprises a protected amino group and a 5-member heteroaromatic or a 6-member aromatic or heteroaromatic ring, the oligomer having a formula (10), (11) or (12):

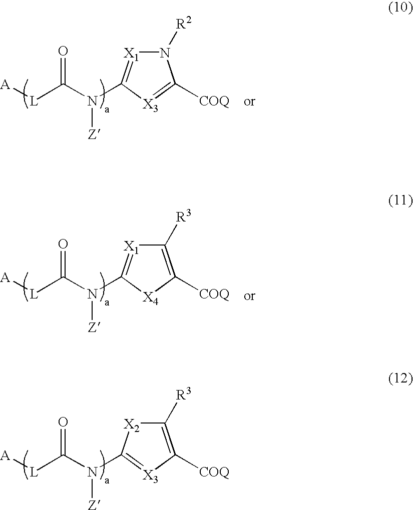

wherein a is at least about 1 and Q, A, Z, Z′, X₁, X₂, X₃, X₄, R¹, R¹′, R², R³, R⁴ and L are broadly defined as noted with respect to formulas (1), (2) and (3), respectively. More preferably, however:

Q is Cl or Br;

a is an integer of at least about 1, and typically is an integer ranging from about 1 or 2 to about 10 (e.g., about 1 to about 10, about 2 to about 8, or about 3 to about 5) and represents the number of tandem units present in the oligomer;

A is H or NZZ′;

Z and Z′ are independently hydrogen or an amine protecting group which is the same or different, provided that at least one of Z and Z′, together with the nitrogen atom to which they are attached, form a carbamate moiety;

X₁ is N or CR¹;

X₂ is O, S, NR¹, —CR¹═CR¹′—, —CR¹═N—, —N═CR¹— or —N═N—;

X₃ is N or CR⁴;

X₄ is O or S;

R¹ and R¹′ are independently selected from hydrogen and substituted or unsubstituted alkyl;

R² is independently selected from hydrogen, substituted or unsubstituted C₁-C₁₀ alkyl, substituted or unsubstituted C₂-C₁₀ alkenyl, or substituted or unsubstituted C₂-C₁₀ alkynyl;

R³ is independently selected from hydrogen, substituted or unsubstituted C₁-C₁₀ alkyl, substituted or unsubstituted C₂-C₁₀ alkenyl, substituted or unsubstituted C₂-C₁₀ alkynyl or halo;

R⁴ is independently selected from hydrogen, hydroxy or alkoxy; and,

L is a subunit of the tandem unit independently selected from substituted or unsubstituted alkylene, substituted or unsubstituted arylene and substituted or unsubstituted heteroarylene, wherein (i) L may be the same or different for each tandem unit, and (ii) no more than about one L is present which enables a hairpin turn therein.

In some preferred embodiment L is selected from: (i) substituted or unsubstituted ethylene (e.g., —CH₂CH₂— or alkoxyethylene, —CH₂CH(OR)—, wherein R is hydrogen or substituted or unsubstituted alkyl (e.g., methyl, ethyl, etc.)); (ii) substituted or unsubstituted propylene (e.g., —CH₂CH₂CH₂—; aminopropylene (—CH₂CH₂CH(NZZ′)—); and, (iii) a substituted or unsubstituted, 5-member heteroarylene or 6-member arylene or heteroarylene ring having a formula:

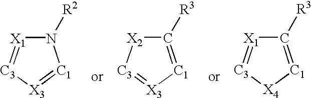

wherein X₁, X₂, X₃, X₄, R² and R³ are as defined above, and further wherein the ring has a 1,3-bond orientation within the tandem unit, one of the C₁ or C₃ carbon atoms in the ring adjacent to X₃ or X₄ being bound to the nitrogen atom in the tandem unit and the other being bound to the carbonyl carbon in the tandem unit.

In this regard it is to be noted that “C₁” and “C₃” in the above ring structures are provided only for the purpose of indicating location of the amino and acid halide substituents on the ring, relative to each other. Accordingly, they are not intended to necessarily indicate the proper numbering of the ring and, as such, should not be viewed in a limiting sense.

It is to be further noted that preferably only one subunit, L, is present which allows the formation of a hairpin (e.g., a beta or gamma) turn; that is, it is preferred that only one of the subunits present is other than unsubstituted ethylene, arylene or heteroarylene (a hairpin turn being formed from, for example, by alkoxylethylene, propylene, aminopropylene, etc.).

It is to be still further noted that the preferred embodiments set forth above with respect to the central ring of the amino acid halide monomer, as well as the substituents attached thereto, also apply here; that is, it is to be noted that the preferred configurations noted above with respect to the monomeric amino acid halide of the present invention also apply, as appropriate, to some of the preferred oligomeric amino acid halides of the present invention. More specifically, in some preferred embodiments the oligomer comprises a 5-member heterocyclic ring, and more preferably has a formula (16A), (16B), (18A) or (18B):

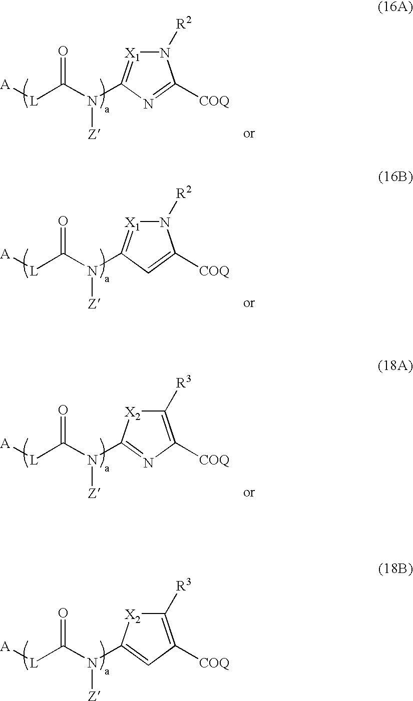

wherein: a, A, Q, Z, Z′, X₁, R¹, R², R³ and L are as previously defined and X₂ is O, S or NR¹.

Among the additional, preferred embodiments are those wherein a in an integer in the range of about 1 to about 10 or about 2 to about 8 and: for formula (17A or B), those wherein Z′ is H; Z is Boc or Fmoc, and more preferably is Boc; Q is chloro or bromo, and more preferably is chloro; X₁ is CH and R² is H or CH₃; and, for formula (19A or B), those wherein Z′ is H; Z is Boc or Fmoc, and more preferably is Boc; Q is chloro or bromo, and more preferably is chloro; X₂ is CH and R³ is H or CH₃.

It is to be still further noted that, in alternative embodiments, the protected amino acid halide oligomer comprises a 6-member aromatic or heteroaromatic ring. Among the preferred 6-member ring structures from which the oligomeric amino acid halides of the present invention are derived include those selected from, for example, substituted or unsubstituted pyridine, substituted or unsubstituted pyrimidine, substituted or unsubstituted pyridazine, substituted or unsubstituted pyrazine, substituted or unsubstituted 1,2,4-triazine, and substituted or unsubstituted benzene, wherein for each the amino and acid halide substituents are separated by a single carbon atom or heteroatom (i.e., the two substitutents have a 1,3 positional relationship within the ring).

II. Acid Halide Preparation

In another embodiment, the present invention is directed to an efficient method for the preparation of amino acid halides, as described herein, using an appropriate carboxylic or thiocarboxylic acids and am α-haloenamine (e.g., an α-chloroenamine or α-bromoenamine), which may or may not be immobilized on a support. Experience to-date indicates such a method is preferred because it enables the preparation of the desired amino acid halides in high purity, the purity typically ranging from about 95 wt % to about 99.9 wt % (e.g., a purity of at least about 95 wt %, 96 wt %, 97 wt %, 98 wt %, 99 wt %, 99.5 wt % or even 99.9 wt %) and high yield, the yield typically ranging from at least about 90% to about 99% (e.g., at least about 90%, 92%, 94%, 96%, 98%, 99% or more).

The preparation and use of α-haloenamines is described below, and in further detail in U.S. patent application Ser. No. 10/061,617, filed on Feb. 1, 2002, which is incorporated in its entirety herein by reference.

A. Preparation of α-Haloenamines

In accordance with one aspect of the present invention, α-haloenamines may be prepared from tertiary amides and pentavalent phosphorous halides. The tertiary amide reacts with the pentavalent phosphorous halide to produce a haloiminium salt which is then converted to the α-haloenamine with a base.

In general, the tertiary amide may be any tertiary amide having a hydrogen atom bonded to the carbon which is in the alpha position relative to the carbonyl group of the tertiary amide and which does not interfere with the synthesis of or react with the α-haloenamine. In one approach, the tertiary amide has the general formula:

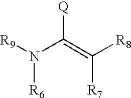

wherein:

R₆ and R₉ are independently hydrocarbyl, substituted hydrocarbyl, hydrocarbyloxy or substituted hydrocarbyloxy;

R₇ and R₈ are independently hydrogen, hydrocarbyl, substituted hydrocarbyl, hydrocarbylthio, substituted hydrocarbylthio, hydrocarbylcarbonyl, substituted hydrocarbylcarbonyl, hydrocarbyloxycarbonyl, substituted hydrocarbyloxycarbonyl, phosphinyl, thiophosphinyl, sulfinyl, sulfonyl, halo, cyano, or nitro; and,

Q is halo, and preferably is chloro or bromo, and more preferably is chloro;

provided that, when the α-haloenamine is immobilized, at least one of R₆, R₇, R₈ and R₉ comprises a support which enables physical separation of the reagent from a liquid mixture.

In at least some embodiments it is preferred that R₇ and R₈ are other than hydrogen, such as alkyl or aryl, to increase the stability of the reagent to a variety of conditions. Nevertheless, under some circumstances, provided one of R₇ and R₈ is sufficiently electron-withdrawing, the other may be hydrogen. Under other circumstances, each of R₇ and R₈ is electron withdrawing. In no event, however, may R₇ and R₈ each be hydrogen.

In one instance, one of R₆, R₇, R₈ and R₉ comprises a support which enables physical separation of the tertiary amide (or a derivative thereof) from a liquid mixture. The support may be, for example, any solid or soluble, organic or inorganic support which is conventionally used in chemical synthesis or any of a variety of assays. Such supports are described in greater detail elsewhere herein in connection with the supported α-haloenamine reagents. Preferably, it is polystyrene or a derivative thereof, for example, a 1% cross linked polystyrene/divinyl benzene copolymer.

The pentavalent phosphorous halide comprises at least two halogen atoms bonded to a pentavalent phosphorous atom. The three remaining valences are optionally occupied by bonds to carbon or halogen atoms. In general, therefore, the pentavalent phosphorous halide may be represented by the general formula P(Q)₂(E)₃ wherein each Q is independently a halogen atom (e.g., chloro or bromo) and each E is independently a halogen atom or a carbon atom (which is part of a hydrocarbyl or substituted hydrocarbyl radical). For example, included within this general formula are pentavalent phosphorous halides in which the pentavalent phosphorous atom is bonded to two, three, four, or five halogen atoms selected from among chlorine, bromine and iodine. If fewer than five halogen atoms are bonded to the pentavalent phosphorous atom, the remaining valences are occupied by phosphorous-carbon bonds with the carbon being part of a hydrocarbyl or substituted hydrocarbyl radical, preferably phenyl or lower alkyl (e.g., methyl, ethyl or isopropyl). Although mixed halides are theoretically possible and within the scope of the present α-haloenamines, for most applications it will generally be preferred that halogen atoms of only one type be attached to the pentavalent phosphorous atom. Phosphorous pentachloride and phosphorous pentabromide are particularly preferred.

The α-haloiminium salt resulting from the reaction of the tertiary amide and the pentavalent phosphorous compound may be converted to the α-haloenamine with an amine base such as N,N-dialkyl anilines, trialkylamines, heterocyclic amines, pyridines, N-alkylimidazole, DBU and DBN. Tertiary amine bases such as triethylamine are generally preferred; other amine bases, however, such as substituted pyridines may be preferred under certain circumstances.

In general, therefore, α-haloenamines may be prepared in accordance with the following reaction scheme:

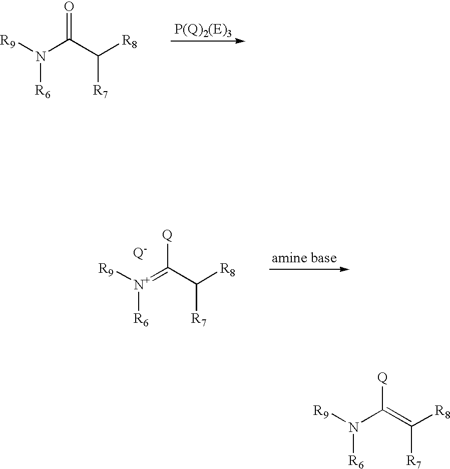

wherein

R₆ and R₉ are independently hydrocarbyl, substituted hydrocarbyl, hydrocarbyloxy, or substituted hydrocarbyloxy;

R₇ and R₈ are independently hydrogen, hydrocarbyl, substituted hydrocarbyl, hydrocarbylthio, substituted hydrocarbylthio, hydrocarbylcarbonyl, substituted hydrocarbylcarbonyl, hydrocarbyloxycarbonyl, substituted hydrocarbyloxycarbonyl, phosphinyl, thiophosphinyl, sulfinyl, sulfonyl, halo, cyano, or nitro, and

each Q is independently chlorine or bromine; and

each E is independently chlorine, bromine, iodine, hydrocarbyl or substituted hydrocarbyl.

The reaction may be carried out in acetonitrile, another solvent, or a mixture of solvents in which pentavalent phosphorous and the tertiary amide are sufficiently soluble. Other solvents include ethereal solvents (e.g., tetrahydrofuran, and 1,4-dioxane), esters (e.g., ethyl acetate), halogenated solvents (e.g., methylene chloride, chloroform and 1,2-dichloroethane), and under certain conditions, hydrocarbon solvents (e.g., toluene and benzene). If the solvent system comprises a mixture of solvents, the solvent system preferably comprises at least about 10% by weight, more preferably at least about 20% by weight acetonitrile.

If desired, the halogen atom, Q, of the resulting α-haloenamine may be displaced by another halogen atom to form other α-haloenamine derivatives. Thus, for example, the chlorine atom of an α-chloroenamine may be displaced by a bromide, fluoride or iodide atom. Similarly, the bromine atom of an α-bromoenamine may be displaced by a fluoride or iodide atom. In general, the displacement may be carried out with an alkali metal halide (e.g., sodium, potassium, cesium or lithium bromide, fluoride or iodide).

B. Immobilized α-Haloenamine Reagents

An immobilized α-haloenamine reagent comprises an α-haloenamine component tethered to a support which enables physical separation of the reagent from a liquid composition. The α-haloenamine component is tethered to the support by means of a linker and, optionally, a spacer. Such immobilized α-haloenamine reagents, suitable for use in the present invention, generally correspond to the formula:

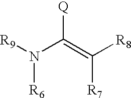

wherein Q is halogen, and R₆, R₇, R₈ and R₉ are as previously defined provided, however, at least one of R₆, R₇, R₈ and R₉ comprises a support which enables physical separation of the reagent from a liquid composition. In general, reactivity tends to be greater when R₆, R₇, R₈ and R₉ are less bulky and when R₆, R₇, R₈ and R₉ are alkyl or aryl. Preferably, therefore, R₆, R₇, R₈ and R₉ are independently hydrocarbyl or substituted hydrocarbyl, more preferably hydrocarbyl, still more preferably alkyl or aryl, provided at least one of R₆, R₇, R₈ and R₉ comprises a support which enables physical separation of the reagent from a liquid composition.

In one embodiment, the α-haloenamine reagent support is a solid which is insoluble under all pertinent conditions. In another embodiment, the haloenamine reagent support is a composition which is selectively soluble in a solvent system; under a first set of conditions, the support is soluble but under a second set of conditions, the support is insoluble.

Insoluble polymers and other solid supports are typically the more convenient form since they may be easily separated from liquids by filtration.

Such supports are routinely used in chemical and biochemical synthesis and include, for example, any insoluble inorganic or organic material that is compatible with chemical and biological syntheses and assays such as glasses, silicates, cross-linked polymers such as cross-linked polystyrenes, polypropylenes, polyacrylamides, polyacrylates and sand, metals, and metal alloys. For example, the α-haloenamine reagent support may comprise poly(N,N-disubstituted acrylamide), e.g., poly(N,N-dialkyl substituted acrylamide) or a copolymer thereof. Preferred materials include polystyrene-based polymers and copolymers. Commercially available materials include TentaGel resin and ArgoGel (Bayer), both polystyrene/divinylbenzene-poly(ethylene glycol) graft copolymers (with ˜1-2% cross-linking) and 1% cross-linked polystyrene/divinylbenzene copolymer (ACROS) available in a range of particle sizes (e.g., 200-400 mesh).

In general, solid supports may be in the form of beads, particles, sheets, dipsticks, rods, membranes, filters, fibers (e.g., optical and glass), and the like or they may be continuous in design, such as a test tube or micro plate, 96 well or 384 well or higher density formats or other such micro plates and micro titer plates. Thus, for example, one, a plurality of, or each of the wells of a micro titer plate (96 well, 384 well or greater) or other multi well format substratum may have the α-haloenamine reagent tethered to its surface. Alternatively, beads, particles or other solid supports having an α-haloenamine reagent of the present invention bound to its surface may be added to one, a plurality of, or each of the wells of a micro titer plate or other multi well substratum. Furthermore, if the solid support (whether in the form of a bead, particle, multi well micro titer plate, etc.) comprises poly(N,N-disubstituted acrylamide) or another polymer having tertiary amides chemically accessible at its surface, these tertiary amides may be converted to immobilized α-haloenamines using a pentavalent phosphorous halide as otherwise described herein; stated another way, the source of the tertiary amide, from which the immobilized α-haloenamine is derived, may simply be a polymeric material comprising chemically accessible tertiary amides.

Solid-phase, polymer bound reagents, however, are not without their shortcomings. For example, phase differences obtained by heterogeneous, insoluble supports can create diffusion limitations due to the polymer matrix and this, in turn, can lead to reduced reactivity and selectivity as compared to classical, solution-phase synthesis. Furthermore, the insoluble nature of these supports can make synthesis and characterization of the polymer-reagent complex difficult. Accordingly, selectively soluble supports are preferred for some applications.

In general, any polymeric material which is soluble under one set of conditions and insoluble under a second set of conditions may be used as a selectively soluble support, provided this group does not interfere with the synthesis of or react with any of the reaction products or intermediates. Exemplary soluble polymers include linear polystyrene, polyethylene glycol, and their various polymers and copolymers derivatized with tertiary amides which may then be converted to α-haloenamines. In general, however, polyethylene glycol is preferred. Polyethylene glycol exhibits solubility in a wide range of organic solvents and water but is insoluble in hexane, diethyl ether, and tert-butyl methyl ether. Precipitation using these solvents or cooling of polymer solutions in ethanol or methanol yields crystalline polyethylene glycol which can be purified by simple filtration. Attaching a haloenamine group to the polyethylene glycol thus allows for homogeneous reaction conditions while permitting for relatively easy purification.

The α-haloenamine functionality or component of the α-haloenamine reagent is preferably attached to the support by means of a linker. The only requirement is that the linker be able to withstand the conditions of the reaction in which the haloenamine reagent will be employed. In approach, the linker is selectively cleavable under a set of conditions to permit cleavage of the enamine from the support. In another approach, the linker is not.

A great number of cleavable linkers have been developed over the years to allow many multi-step organic syntheses to be performed. These linkers have generally been classified into several major classes of cleavage reaction (with some overlap between classes): (a) electrophilically cleaved linkers, (b) nucleophilically cleaved linkers, (c) photocleavable linkers, (d) metal-assisted cleavage procedures, (e) cleavage under reductive conditions, and (f) cycloaddition- and cycloreversion-based release. (See, e.g., Guillier et al., Linkers and Cleavage Strategies in Sold-Phase Organic Synthesis and Combinatorial Chemistry, Chem. Rev. 2000, 100, 2091-2157.)

More typically, the linker is non-cleavable and merely constitutes a chain of atoms connecting the α-haloenamine to the solid support. The only requirement is that the sequence not react with any of the final products or intermediates. Thus, for example, any of the standard chemistries used to attach molecules to a solid support may be used to immobilize the α-haloenamine or, more preferably, a tertiary amide precursor which is then converted to the α-haloenamine using a pentavalent phosphorous halide. More specifically, a solid phase α-chloroenamine reagent may be derived from a polystyrene supported tertiary amide and PCl₅, with the polystyrene supported tertiary amide, in turn, being derived from polystyrene and a chloro-substituted tertiary amide in the presence of FeCl₃ (see Example 2). Alternatively, styrene (or another polymerizable monomer) having a tertiary amide as a substituent on the phenyl ring may be polymerized to form a polymer having a pendant tertiary amide which, as described elsewhere herein, may be converted to an α-haloenamine moiety using a pentavalent phosphorous halide, followed by treatment with a base.

Regardless of whether the linker is cleavable or non-cleavable, it may optionally include a spacer having a length and/or included moieties which provide the α-haloenamine reagent with more “solution-like” properties and better solvent compatibility. In general, the spacer group, if present, may be any atom, or linear, branched, or cyclic series of atoms which distance the α-haloenamine group from the support. The atoms, for example, may be selected from carbon, oxygen, nitrogen, sulfur and silicon. Preferred spacers include polyethylene glycol and alkyl chains.

In one embodiment of the α-haloenamine, one of R₆ and R₉ comprises a support and R₇, R₈ and the carbon atom to which they are attached are members of a carbocylic or heterocyclic ring:

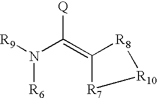

wherein R₆, R₇, R₈, R₉ and Q are as previously defined and R₁₀ is an atom or chain of atoms, which together with R₇ and R₈ define a carbocyclic or heterocyclic structure. If the structure is heterocyclo, the hetero atoms are preferably selected from oxygen and sulfur; basic nitrogens are preferably not included as a ring atom. In addition, the atom or chain of atoms comprising R₁₀ may be substituted with one or more hydrocarbyl, substituted hydrocarbyl, hetero atom(s) or heterocyclo substituent. For example, together R₇, R₈ and R₁₀, along with the carbon atom to which R₇ and R₈ are attached, may comprise a cycloalkyl ring such as cyclopentyl or a five- or six-member heterocyclic ring. In another embodiment, R₈ comprises a support which enables physical separation of the reagent from a liquid composition, and any two of R₆, R₇ and R₉ and the atoms to which they are attached are members of a heterocyclic ring:

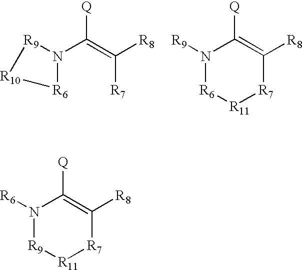

wherein R₆, R₇, R₈, R₉ and Q are as previously defined and R₁₀ is an atom or a chain of atoms, and R₁₁ is a bond, an atom or chain of atoms, wherein R₁₀, together with R₆ and R₉, or R₁₁ together with R₆ and R₇, or R₇ and R₉ define a carbocyclic or heterocyclic structure. If the ring is heterocyclo, the hetero atoms are preferably selected from oxygen and sulfur; again, basic nitrogens are preferably not included as a ring atom. In each of these embodiments, R₁₀ preferably comprises two or three chain atoms selected from carbon, oxygen and sulfur, and R₁₁ is preferably a bond or an atom selected from carbon, oxygen and sulfur, thereby defining in each instance, a 5- or 6-membered heterocycle. In addition, the atom or chain of atoms or which R₁₀ and R₁₁, are comprised may optionally be substituted with one or more hydrocarbyl, substituted hydrocarbyl, hetero atom(s) or heterocyclo substituents.

In this regard it is to be noted that, with respect to the previously described embodiments wherein, for example, two of R₆, R₇, R₈, R₉, together with the atoms to which they are attached, define a carbocyclic or heterocyclic ring, the unsaturated bonds present in said ring will have a cis configuration.

C. Reaction Conditions

The α-haloenamines described herein and, in one preferred embodiment, the immobilized α-haloenamines, may be used to convert a wide variety of carboxylic and thiocarboxylic acids to the corresponding acid halides. However, to avoid or at least minimize unwanted side reactions, these acids preferably have an absence of other unprotected moieties which are also reactive with α-haloenamines (e.g., hydroxy, thio, amino, etc. substituents). For example, basic primary and secondary amine moieties will react with α-haloenamines and thus, it is preferred that the carboxylic or thiocarboxylic acid compounds have an absence of unprotected basic primary and secondary amine moieties when it is reacted with an α-haloenamine of the present invention. As previously noted, suitable protecting groups are identified, for example, in Protective Groups in Organic Synthesis by T. W. Greene and P. G. M. Wuts, John Wiley and Sons, 3rd ed. 1999.

In accordance with the present invention, an α-haloenamine is used to convert any of a wide range of carboxylic acids and thiocarboxylic acids to the corresponding acid halide monomer or oligomer. More specifically, in one embodiment, an immobilized α-haloenamine as described herein and a 5-member heteroaromatic, or 6-member aromatic or heteroaromatic, carboxylic acid or thiocarboxylic acid, which has a protected amino substituent attached thereto, are contacted to form the corresponding 5-member or 6-member protected amino acid halide of the present invention.

In this regard it is to be noted that “corresponding” as used herein means the acid and acid halide essentially differ only by the group attached to the carbonyl carbon. For example, a protected amino acid chloride of the present invention and the carboxylic acid from which it is derived will essentially differ only in that Q is chloro in the former and hydroxy in the latter. Accordingly, the carboxylic or thiocarboxylic acids suitable for use in the present invention may be generally represented by formula (1), (2) or (3), with the exception that Q is —OH or —SH rather that halo.

Some exemplary carboxylic acids have a formula such as:

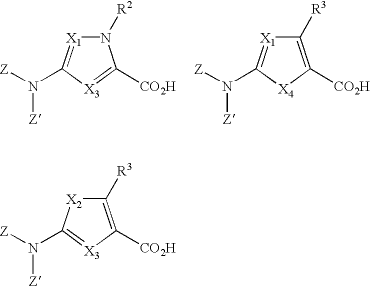

wherein Z, Z′, X₁, X₂, X₃, X₄, R¹, R², R³ and R⁴ are as defined herein with respect to formula (4), (5) and (6), with X₃ in some preferred embodiments being nitrogen or CH (i.e., having a formula corresponding to formulas (7A), (9A) or (7B), (9B), respectively, as described above, with the exception that in this instance Q is —OH rather than chloro or bromo). Alternatively, however, oligomeric acids (e.g., carboxylic acids) may be used, such as those having a formula:

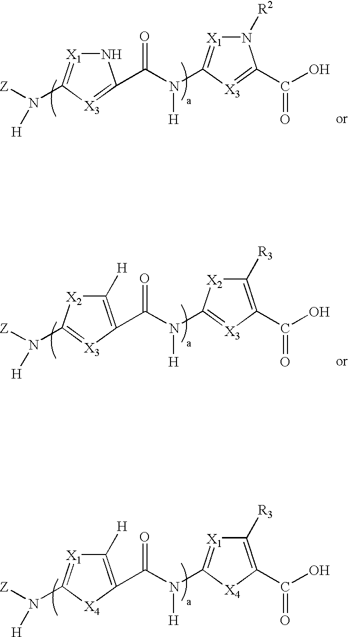

wherein, for example, Z′ is H, and a, Z, X₁, X₂, X₃, X₄, R¹, R², R³ and R⁴ are as defined herein with respect to formula (10), (11) or (12).

Generally speaking, the reaction to prepare the amino acid halides of the present invention may be carried out using means common in the art; that is, an α-haloenamine and an appropriate carboxylic or thiocarboxylic acid may be brought into contact to react using means known in the art. Typically, however, these reagents are contacted in a ratio of about 1:1, although in some instances a slight excess of the α-haloenamine will be used (e.g., a ratio within the range of about 1:1 to about 2:1 α-haloenamine to acid, about 1.2:1 to about 1.8:1, or even about 1.4:1 to about 1.6:1). Optionally, these reagents will be contacted in the presence of a suitable solvent (e.g., acetonitrile or dimethylformamide (DMF)), the concentration of the acid in solution ranging from about 0.01 moles/liter to about 1 mole/liter, from about 0.05 moles/liter to about 0.5 moles/liter, or from about 0.075 moles/liter to about 0.25 moles/liter. The resulting reaction mixture is maintained at room temperature for less than about 30 minutes, about 20 minutes, about 10 minutes, about 5 minutes or even about 1 minute, in order to obtain the resulting acid halide, with the reaction time typically ranging from about 1 to about 10 minutes. Although the temperature and pressure of the reaction may be controlled, for example, to impact the reaction time, typically the reaction mixture is maintained at about room temperature (e.g., about 20° C. to about 30° C.) and at ambient pressure for the duration of the reaction. Alternatively, however, the temperature of the reaction mixture may be in the range of about 25° C. to about 100° C., or about 30° C. to about 75° C.

III. Polyamide Preparation

A. Overview

Once the amino acid halides of the present invention have been prepared, they may be used by means common in the art to prepare, isolate and purify the desired polyamide of interest. Such processes may be carried out in solution phase or solid phase, with solid phase being preferred in at least some embodiments because of the added ease of isolation and purification of the resulting polyamide.

Generally speaking, the solid phase polyamide synthesis protocols employed may be, for example, like those disclosed in U.S. Pat. No. 6,090,947 (the entire contents of which is incorporated herein by reference). (See also Baird et al., J. Am. Chem. Soc., 1996, 118, 6141-46.) Briefly, the method of preparing, for example, imidazole and pyrrole carboxamide polyamides according to the present invention may be generally defined to include the following series of steps:

(1) The appropriate amino acid monomer or oligomer (e.g., dimer, trimer, etc.) is protected at the amino group and activated at the carboxylic acid group. The amino group is preferably protected with a Boc or Fmoc protecting group and the carboxylic acid is activated by the formation of the acid halide (e.g., chloride) as described herein, to yield, in the case of the pyrrole and imidazole, the amino acids Boc-pyrrole-COCl, Boc-imidazole-COCl, Fmoc-pyrrole-COCl and Fmoc-imidazole-COCl.

(2) Beginning with the acid halide terminal, Boc-protected and activated amino acid halides are then sequentially added, for example, to a commercially available solid support (e.g., polystyrene resin) which has previously been treated, as necessary, in order to liberate the protected amino group attached thereto. Traditionally, high concentrations of activated monomer (i.e., a 4:1 ratio of monomer to unprotected, growing polymer chain) are utilized in an attempt to increase the rate at which the coupling reactions occur. However, according to in the present invention, a much lower ratio may be employed, as further described herein.

In situ neutralization chemistry is utilized in conjunction with the acid catalyzed removal of Boc protecting groups (which otherwise results in the formation of a protonated amine), to assure that an unstable, deprotonated amine is generated simultaneous with the initiation of a coupling reaction. (As further described by, for example, Dervan et al. in U.S. Pat. No. 6,090,947.) For conventional techniques, coupling times are typically over an hour per residue. However, according to the present invention and as further described herein, such coupling reactions generally take less than about 30 minutes to complete.

(3) When the desired polyamide has been prepared, the amino acids are deprotected and the peptide is cleaved from the resin and purified using techniques common in the art. The reactions may be periodically monitored using, for example, picric acid titration and high pressure liquid chromatography (as further described by, for example, Dervan et al. in U.S. Pat. No. 6,090,947).

B. Polyamide Synthesis Using Protected Amino Acid Halides

Accordingly, in one embodiment the present invention is directed to a process for preparing a polyamide oligomer or polymer which comprises forming a population of amino-protected, amino acid halide building block units possessing a 5-member heteroaromatic or 6-member aromatic or heteroaromatic moiety therein, the units being formed by contacting an immobilized α-haloenamine and a corresponding, amino-protected, 5-member heteroaromatic or 6-member aromatic or heteroaromatic carboxylic acid. A member of the population of amino-protected, amino acid halide building block units is then combined with a moiety comprising an unprotected amino functionality to form a reaction product possessing an amide linkage and a protected amino functionality. In the case of solid phase synthesis, the moiety comprising the unprotected amino functionality is immobilized on a solid support, such as by covalent bonding.

Once the reaction has occurred, the amino group of the reaction product is deprotected, using techniques known in the art, and then it is combined with another amino-protected, amino acid halide building block. This sequence of steps is continued until the desired length or molecular weight of the polymer or oligomer has been achieved. Finally, the remaining protecting group is removed, and then the polymer or oligomer is cleaved from the support (if one is present) and purified using means common in the art. Such a reaction may be generally represented by the scheme (using formula (4) to illustrate):

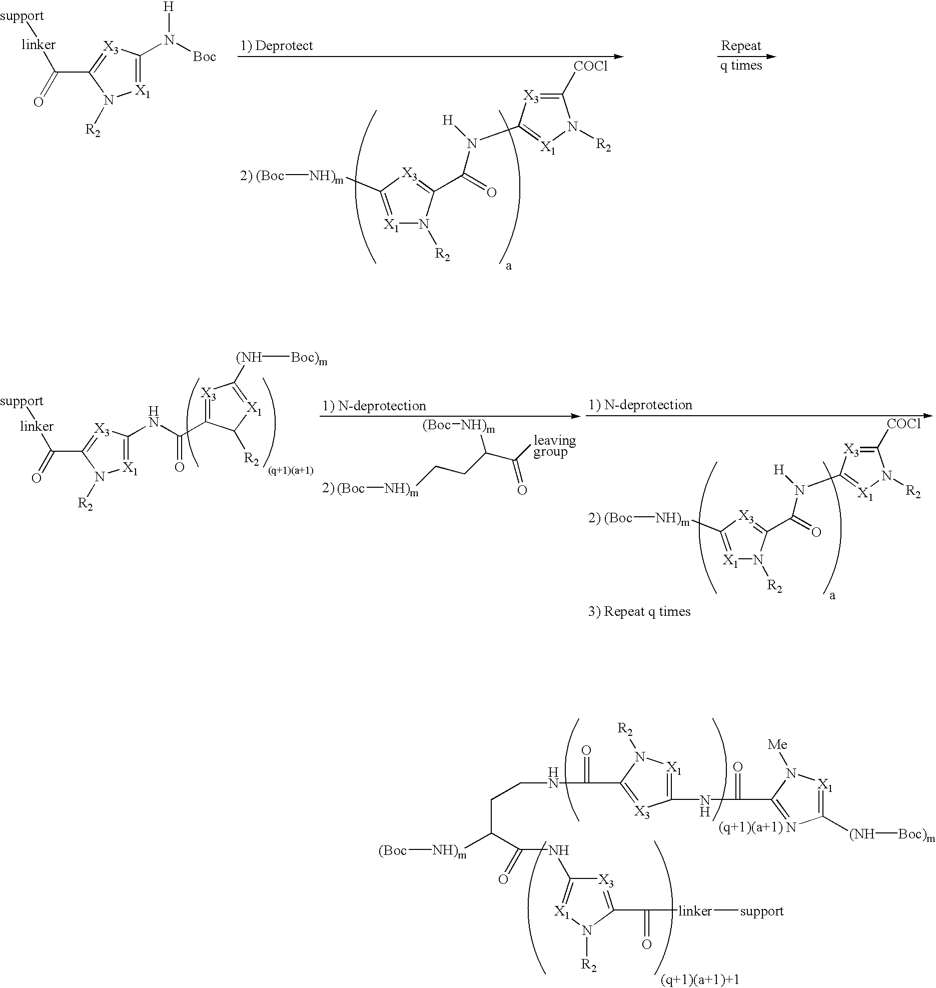

As previously noted, the present process is advantageous over existing methods, at least in part because the amino acid halide is very reactive. As a result, the reaction process proceeds further toward completion, thus improving efficiency and yield. In addition, the reactivity of the amino acid halide enables lesser amounts of the reagent to be employed. For example, unlike conventional processes, wherein a 4:1 ratio of amino acid monomer to unprotected polymer or oligomer is used, the present process enables a ratio of equivalents of the amino-protected, amino acid halide building block units to the moiety comprising an unprotected amino functionality to be within a range of about 1:1 to less than about 4:1, or from about 1:1 to about 3:1, the ratio preferably being about 3:1, more preferably about 2.5:1, still more preferably about 2:1, still more preferably about 1.5:1 or even most preferably about 1:1. The present process enables the efficient production of polyamides in high yield, the yield typically being within the range of about 90% to about 100%, and preferably being at least about 92%, 94%, 96%, or even 98%.

The process of preparing polyamides maybe also be carried out under conditions known in the art, including room temperature and ambient pressure, the reaction to add each monomer typically being completed in less than about 30 minutes, 20 minutes, 10 minutes, 5 minutes or even 1 minute, with reaction times of about 1 to about 20 minutes or about 5 to about 10 mintues being typical in at least some embodiments.

In one preferred embodiment, the monomer utilized in the polyamide synthesis of the present invention has a formula (4), (5) or (6), and more preferable has a formula (7A), (7B), (9A) or (9B). Among the particularly preferred monomers for this embodiment are those previously noted with respect to these formulas, as well as those wherein Q is chloro, Z is Boc or Fmoc and: (i) X₁ is N or CH, X₃ is N or CH, and R² is hydrogen or methyl, (ii) X₁ is N or CH, X₄ is O or S, and R³ is hydrogen or methyl, or (iii) X₂ is S, O or NR¹, X₃ is N or CH, and R¹ and R³ are hydrogen or methyl.

IV. Definitions

The terms “hydrocarbon” and “hydrocarbyl” as used herein describe organic compounds or radicals consisting exclusively of the elements carbon and hydrogen. These moieties include linear, branched or cyclic alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms. In addition, with respect to the tertiary amide or α-haloenamine described herein, the hydrocarbyl moiety may be linked to more than one substitutable position; for example, R₇ and R₈ of the tertiary amide or α-haloenamine may comprise the same chain of carbon atoms which, together with the carbon atoms to which R₇ and R₈ are attached define a carbocyclic ring.

The “substituted hydrocarbyl” moieties described herein are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, heterocyclo, alkoxy, alkenoxy, alkynoxy, aryloxy, hydroxy (preferably on aryl or heteroaryl rings), protected hydroxy, formyl, acyl, acyloxy, amino, amido, nitro, cyano, thiol, sulfides, sulfoxides, sulfonamides, ketals, acetals, esters and ethers.

The term “heteroatom” shall mean atoms other than carbon and hydrogen.

Unless otherwise indicated, the alkyl groups described herein are preferably lower alkyl containing from about one to about eight or ten carbon atoms in the principal chain, and up to about 20 carbon atoms. They may be straight or branched chain or cyclic and include methyl, ethyl, propyl, isopropyl, butyl, pentyl, hexyl and the like.

Unless otherwise indicated, the alkenyl groups described herein are preferably lower alkenyl containing from about two to about eight or ten carbon atoms in the principal chain, and up to about 20 carbon atoms. They may be straight or branched chain or cyclic and include ethenyl, propenyl, isopropenyl, butenyl, isobutenyl, pentenyl, hexenyl, and the like.

Unless otherwise indicated, the alkynyl groups described herein are preferably lower alkynyl containing from about two to about eight or ten carbon atoms in the principal chain, and up to about 20 carbon atoms. They may be straight or branched chain and include ethynyl, propynyl, butynyl, isobutynyl, pentynyl, hexynyl, and the like.

The terms “aryl” or “ar” as used herein alone or as part of another group denote optionally substituted carbocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The terms “halogen” or “halo” as used herein alone or as part of another group refer to chlorine, bromine, fluorine, and iodine.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group, in some embodiments, preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclo include heteroaromatics such as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, halogen, heterocyclo, alkoxy, alkenoxy, alkynoxy, aryloxy, hydroxy (preferably on aryl or heteroaryl rings), protected hydroxy, formyl, acyl, acyloxy, amino, amido, nitro, cyano, thiol, sulfides, sulfoxides, sulfonamides, ketals, acetals, esters and ethers. In addition, the heterocyclo moiety may be linked to more than one substitutable position of the tertiary amide or α-haloenamine; for example, R₆ and R₇ of the tertiary amide or α-haloenamine may comprise the same chain of atoms which, together with the atoms to which R₆ and R₇ are attached define a heterocyclo ring.

The term “heteroaromatic” as used herein alone or as part of another group denote optionally substituted aromatic groups having at least one heteroatom in at least one ring, and preferably 5 or 6 atoms in each ring. The heteroaromatic group in some embodiments preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, halogen, heterocyclo, alkoxy, alkenoxy, alkynoxy, aryloxy, hydroxy (preferably on aryl or heteroaryl rings), protected hydroxy, formyl, acyl, acyloxy, amino, amido, nitro, cyano, thiol, sulfides, sulfoxides, sulfonamides, ketals, acetals, esters and ethers.

The term “hydrocarbyloxy,” as used herein denotes a hydrocarbyl group as defined herein bonded through an oxygen linkage (—O—), e.g., RO— wherein R is hydrocarbyl.

The term “polyamide” as used herein refers to an oligomer or polymer of heterocyclic amino acids attached to one another by amide linkages (CONH).

The term “amino acid” refers to an organic molecule including both an amino group (NH₂) and a carboxylic acid group (COOH).

The term “amide compound” as used herein refers to a polymer chain of at least two monomer subunits and at least one amide linkage.

The terms “acid halide” and “acid halide derivative,” as used interchangeably herein, refer to derivatives of heterocyclic amino acids that are prepared using a suitable α-haloenamine. In an exemplary embodiment, “halide” and “halo-” refer to compounds containing the halogen atom Cl, but alternatively the halogen atom is Br.

The term “acid halide monomer” as used herein refers to acid halides including an amine group and one carbonyl halide group in which the amine group is protected with a protecting group such as, for example, the Boc protecting group (tert-butoxycarbonyl), or the Fmoc protecting group (9-fluoroenylmethoxycarbonyl).

The term “acid halide oligomer” as used herein refers to acid halides including at least one amide group, one carbonyl halide group, and 0-1 amine groups in which the amine group is protected with a protecting group such as, Boc or Fmoc.

The term “acid halide intermediate” as used herein refers to an acid halide monomer or acid halide oligomer.

The term “tandem unit” as used herein refers to units, which may or may not be identical, in the oligomer which are consecutive or appear one after the other in the larger molecule.

“DBU” shall mean 1,8-diazabicyclo[5.4.0]undec-7-ene.

“DBN” shall mean 1,5-diazabicyclo[4.3.0]non-5-ene.

“Boc” shall mean t-butoxycarbonyl.

“Fmoc” shall mean 9-fluoroenylmethoxy carbonyl.

The following Examples in some instances illustrate a portion of the present invention (e.g., the preparation of a protected amino acid halide and the use thereof to prepare a polyamide oligomer), while in other instances illustrate various features of the α-haloenamine which is used to prepare the protected amino acid halides of the present invention. They are therefore not to be viewed in a limiting sense.

### EXAMPLE 1

### Improved Synthesis of N-(1-chloro-2-methylprop-1-enyl)-N,N-dimethylamine

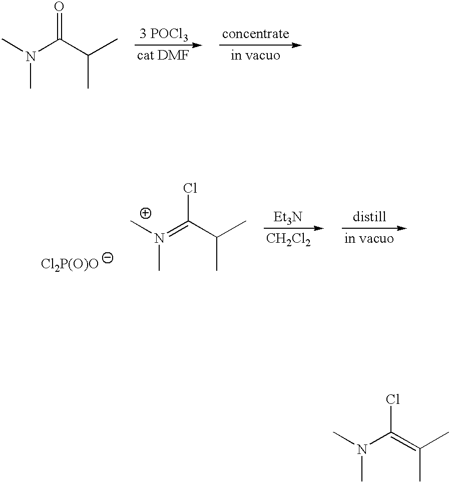

Dimethylisobutyramide (25.00 g, 217.39 mmol) was added dropwise over a 30-minute period to a solution of DMF (336 μL, 4.34 mmol) and POCl₃ (60.70 mL, 651.22 mmol). The resulting solution was stirred at ambient temperature and monitored by ¹H-NMR. After 3 hours the reaction was concentrated under vacuum to remove all excess POCl₃. Triethylamine (33.30 mL, 238.91 mmol) was then added dropwise to a solution of the resulting chloroiminium salt dissolved in a small amount of CH₂Cl₂ (10 mL). This mixture was distilled at 70 C. (100 Torr) to afford 22.70 g of N-(1-chloro-2-methylprop-1-enyl)-N,N-dimethylamine. ¹H NMR (CDCl₃): δ2.36 (s, 6H), 4.11 (s, 2H), 1.74 (br s, 6H).

### EXAMPLE 2

### Synthesis of N-(1-chloro-2-methylprop-1-enyl)-N-methyl aminomethylpolystyrene

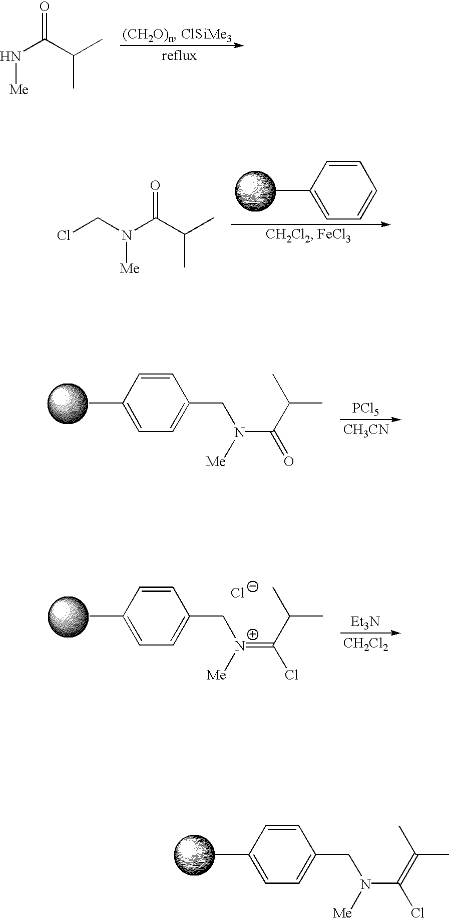

N-Chloromethyl-N-methyl isobutyramide: A mixture of N-methylisobutyramide (200.00 g, 1980 mmol) and paraformaldehyde (50.50 g, 1680 mmol) in chlorotrimethylsilane (860.40 g, 7920 mmol) was slowly heated to reflux. At about 62 C., the reaction exothermed and most of the paraformaldehyde dissolved. This mixture was refluxed for an additional 4 hours, and then was filtered to remove solids. This was concentrated to remove nearly all the excess TMSCI, and then again filtered to afford 219 g of N-chloromethyl-N-methylisobutyramide. ¹H NMR (CDCl₃): (2 rotamers): δ5.33 (s) and 5.30 (s) [2H combined], 3.11 (s) and 2.97 (s) [3H combined], 2.93 (heptet, J=6.2 Hz) and 2.75 (heptet, J=6.4 Hz) [1H combined], 1.14 (d, J=6.2 Hz) and 1.10 (d, J=6.2 Hz) [6H combined].

N-Methyl isobutyramidomethylpolystyrene: Anhydrous FeCl₃ (202.70 g, 1250 mmol) was added in portions to a mechanically stirred mixture of 1% crosslinked styrene-divinylbenzene copolymer (100 g, 960 mEq) and N-chloromethyl-N-methylisobutyramide (186.80 g,1250 mmol) in CH₂Cl₂ (1 L), maintaining the internal reaction temperature between −5° C. to 5° C. The resulting yellow slurry was stirred at room temperature for 5 days, and then was filtered and washed with CH₂Cl₂ (3×), 1:1 aqueous 1N HCl/1,4-dioxane (1×), and then with portions of MeOH until the color was gone. The 1:1 1N HCl/1,4-dioxane wash step was very exothermic and controlled by 1^{st} adding the 1,4-dioxane to the resin, and then cooling this stirred slurry with a dry ice/acetone bath while 1N HCl was added slowly. Vacuum drying at room temperature overnight afforded 193.0 g of the resin as an off-white solid. Amide loading on the resin was calculated to be 4.56 mmol/gm based on elemental analysis. Magic Angle ¹³C NMR (CD₂Cl₂): (2 rotamers): δ177.38 and 176.95 (CO), 53.12 and 50.57 (CH₂N), 34.70 and 34.00 (NCH₃), 30.56 and 30.45 (CHMe₂), 20.03 and 19.53 (CH(CH₃)₂). FT-IR: 1642.92 cm−1 (broad CO stretch). Anal. Calcd for 1.00 C₁₄H₁₉NO+0.10H₂O: C, 76.74; H, 8.83; N, 6.39; 0, 8.03. Found: C, 76.65; H, 8.74; N, 6.30; O, 7.81.

N-(1-Chloro-2-methylprop-1-enyl)-N-methyl aminomethylpolystyrene: N-methyl isobutyramidomethylpolystyrene (100.00 g, 456 mEq) was washed twice with dry CH₃CN (@1.5 L). A fresh portion of CH₃CN (1.5 L) was then added, and the reaction was cooled with an ice-water bath while PCl₅ (330.16 g, 1585 mmol) was added in portions, at a rate which maintained the internal reaction temperature from 10° C. to 17° C. The resulting mixture was slowly stirred at room temperature for 4 hours, and then was filtered and washed with 2 portions of CH₃CN. The swelled polymer was compacted 3-fold by washing with 3 portions of CHCl₃. This CH₃CN/CHCl₃ cycle of washes was repeated to completely remove the excess PCl₅.

A slurry of this chloroiminium chloride of N-methyl isobutyramidomethylpolystyrene was prepared in anhydrous CHCl₃ (1.5 L). This was cooled with dry-ice/acetone to −10° C. while Et₃N (317 mL, 2275 mmol) was added dropwise. A precipitate of Et₃NHCl did not form. The resulting mixture was stirred at 0° C. for 2 hours, and then was filtered and washed sequentially with equal portions of CHCl₃, 1:2 CH₃CN/CHCl₃, 1:1 CH₃CN/CHCl₃, and then CHCl₃. Reaction solvents were anhydrous and the CHCl₃ was stabilized with amylenes. Vacuum drying afforded golden yellow N-(1-chloro-2-methylprop-1-enyl)-N-methyl aminomethylpolystyrene.

Resin loading was determined by adding excess acetic acid (26.9 mg) to a slurry of N-(1-chloro-2-methylprop-1-enyl)-N-methyl aminomethylpolystyrene (96 mg) in CDCl₃ (800 mL), and integrating the acetyl peaks in the 1H NMR spectrum after 10 minutes of stirring at room temperature. A value of 2.64 mEq/gm was calculated from [(CH₃COCl integral)/(CH₃COOH integral)]×26.9 mg/60.05/0.096 g.

### EXAMPLE 3

### Synthesis of 1-methyl-4-(BOC amino)pyrrole-2-carbonyl chloride

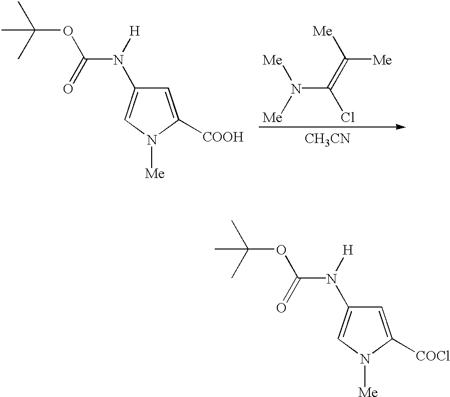

N-(1-chloro-2-methylprop-1-enyl)-N,N-dimethylamine (31 μL, 0.23 mmol) was added to a mixture of 1-methyl-4-(BOC amino)pyrrole-2-carboxylic acid (50 mg, 0.21 mmol) in CDCl₃ (200 mL). After a few minutes, the ¹H-NMR of the reaction mixture showed complete conversion of the acid to the acid chloride. The proton spectrum of this solution of acid chloride did not change on standing overnight at room temperature. ¹H NMR (CDCl₃): δ7.35 (br s, 1H), 6.93 (d, J=2 Hz, 1H), 6.46 (br s, 1H), 3.82 (s, 3H), 1.50 (s, 9H).

### EXAMPLE 4

### Synthesis of 1-methyl-4-(BOC amino)imidazole-2-carbonyl chloride

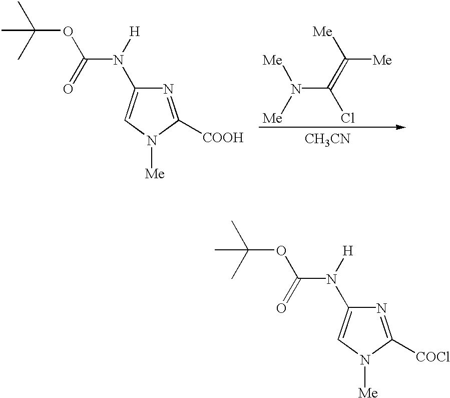

N-(1-chloro-2-methylprop-1-enyl)-N,N-dimethylamine (660 μL, 4.99 mmol) was added to a mixture of 1-methyl-4-(BOC amino)imidazole-2-carboxylic acid (1.00 g, 4.17 mmol) in CHCl₃ (8.00 mL). After a few minutes, the ¹H-NMR of the reaction mixture showed complete conversion of the acid to the acid chloride. ¹H NMR (CDCl₃): δ7.48 (br s, 1H), 3.95 (s, 3H), 1.50 (s, 9H).

### EXAMPLE 5

### General synthesis of acid chlorides using N-(1-chloro-2-methylprop-1-enyl)-N-methyl aminomethylpolystyrene

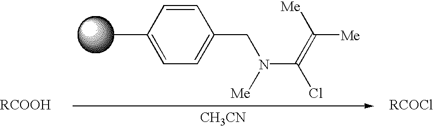

In a dry box, 2 equivalents of N-(1-chloro-2-methylprop-1-enyl)-N-methyl aminomethylpolystyrene was added to a stirred 0.20M-0.25M mixture of -0.5˜1.0 mmol of a carboxylic acid in CD₃CN. The resulting reaction mixture was monitored to completion by ¹H-NMR. Aliquots of the liquid phase containing the acid chloride were then derivatized by addition to small volumes of methanol, ethanol, or aqueous 40% MeNH₂. These reactions were monitored to completion over 1-3 h by ¹H-NMR and HPLC to form the ester or amide, and then were concentrated under vacuum and characterized. Reverse phase HPLC was carried out on an Agilent 1100 system using a Vydac 4.6×250 mm Protein & Peptide C18 column eluted at 1.2 mL/min with a linear gradient of 20% MeCN: 80% H₂O to 100% MeCN over a 15 minute period. Both solvents contained 0.1% TFA. The compounds of examples A-M were prepared by these procedures.

### EXAMPLE 5A

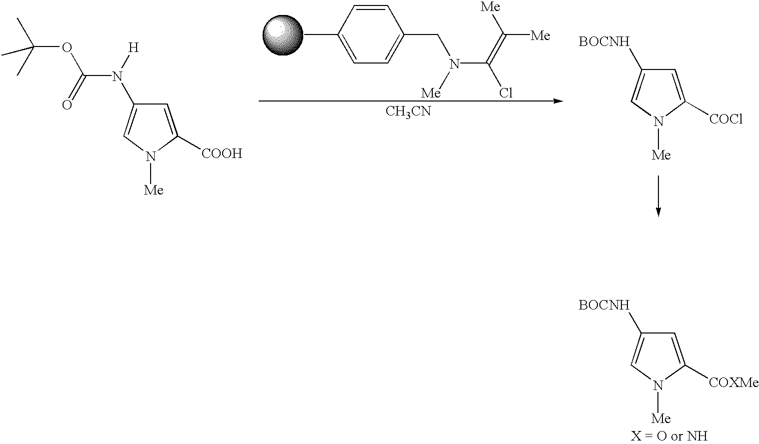

(5-Chlorocarbonyl-1-methyl-1H-pyrrol-3-yl)-carbamic acid tert-butyl ester was cleanly and completely formed from 4-tert-butoxycarbonylamino-1-methyl-1H-pyrrole-2-carboxylic acid overnight. ¹H NMR (CD₃CN): δ7.35 (br s, 1H), 7.03 (d, J=1.9 Hz, 1H), 3.79 (s, 3H), 1.47 (s, 9H). ¹³C NMR (CD₃CN): δ156.20, 125.54, 114.59, 36.96, 27.69.

Methyl 4-tert-butoxycarbonylamino-1-methyl-1H-pyrrole-2-carboxylate formed cleanly and completely from the reaction of (5-chlorocarbonyl-1-methyl-1H-pyrrol-3-yl)-carbamic acid tert-butyl ester with methanol, to afford a single 304 nm HPLC peak at 8.547 min. ¹H NMR (CD₃CN): δ7.27 (br s, 1H), 7.03 (br s, 1H), 6.63 (s, 1H), 3.82 (s, 3H), 3.74 (s, 3H), 1.46 (s, 9H). ¹³C NMR (CD₃CN): δ161.35, 153.37, 123.07, 119.54, 107.68, 50.70 (OCH₃), 36.13, 27.73. Calculated C₁₂H₁₉N₂O₄ (M⁺+1) exact mass=255.1339. Found 255.1333.

(1-Methyl-5-methylcarbamoyl-1H-pyrrol-3-yl)-carbamic acid tert-butyl ester formed cleanly and completely from the reaction of (5-chlorocarbonyl-1-methyl-1H-pyrrol-3-yl)-carbamic acid tert-butyl ester with aqueous 40% methylamine, to afford a single 304 nm HPLC peak at 5.870 min. ¹H NMR (CD₃CN): δ7.27, (br s, 1H), 6.82 (br s, 1H), 6.58 (br s, 1H), 6.45 (s, 1H), 3.81 (s, 3H), 2.76 (d, J=4.7 Hz, 3H), 1.46 (s, 9H). ¹³C NMR (CD₃CN): δ162.25, 153.43, 122.49, 122.34, 102.76, 35.78, 35.75, 27.76, 25.09 (NCH₃). Calculated C₁₂H₂₀N₃O₃ (M⁺+1) exact mass=254.1499. Found 254.1504.

### EXAMPLE 5B

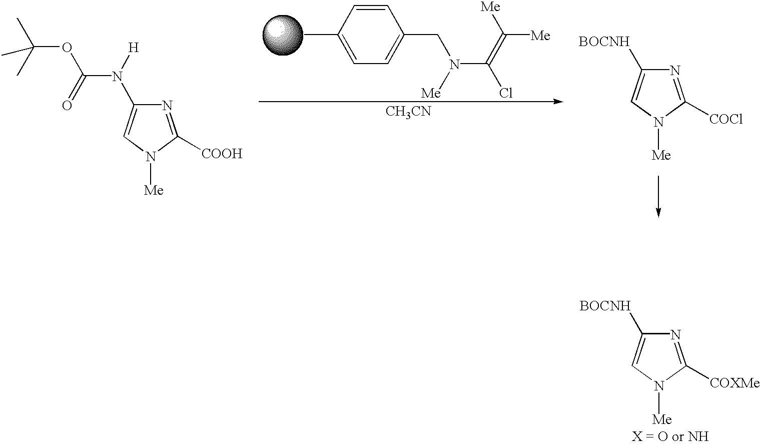

(2-Chlorocarbonyl-1-methyl-1H-imidazol-4-yl)-carbamic acid tert-butyl ester cleanly and completely formed from 4-tert-butoxycarbonylamino-1-methyl-1H-imidazole-2-carboxylic acid within 1 hour. ¹H NMR (CD₃CN): δ8.02 (br s, 1H), 7.50 (br s, 1H), 3.89 (s, 3H), 1.48 (s, 9H).

Methyl 4-tert-butoxycarbonylamino-1-methyl-1H-imidazole-2-carboxylate formed cleanly and completely from the reaction of (2-chlorocarbonyl-1-methyl-1H-imidazol-4-yl)-carbamic acid tert-butyl ester with methanol, to afford a single 304 nm HPLC peak at 6.088 min. ¹H NMR (CD₃CN): δ9.11 (br s, 1H), 7.35 (br s, 1H), 3.98 (s, 3H), 3.92 (s, 3H), 1.49 (s, 9H). Calculated C₁₁H₁₈N₃O₄ (M⁺+1) exact mass=256.1292. Found 256.1291.

(1-Methyl-2-methylcarbamoyl-1H-imidazol-4-yl)-carbamic acid tert-butyl ester formed cleanly and completely from the reaction of (2-chlorocarbonyl-1-methyl-1H-imidazol-4-yl)-carbamic acid tert-butyl ester with aqueous 40% methylamine, to afford a single 304 nm HPLC peak at 5.642 min. ¹H NMR (CD₃CN): δ7.36, (br s, 1H), 7.04 (br s, 1H), 3.94 (s, 3H), 2.81 (s, 3H), 1.47 (s, 9H). Calculated C₁₁H₁₉N₄O₃ (M⁺+1) exact mass=255.1452. Found 255.1429.

### EXAMPLE 6

### Synthesis of 4-[(4-tert-butoxycarbonylamino-1-methyl-1H-imidazole-2-carbonyl)-amino]-1-methyl-1H-imidazole-2-carboxylic acid methyl ester using N-(1-chloro-2-methylprop-1-enyl)-N-methyl aminomethylpolystyrene

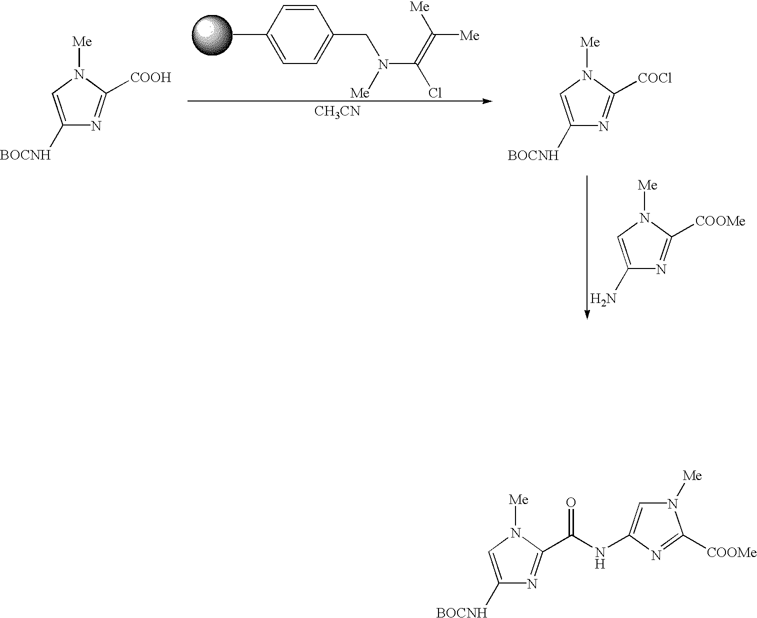

A mixture of 4-tert-butoxycarbonylamino-1-methyl-1H-imidazole-2-carboxylic acid (1.30 g, 5.40 mmol) and N-(1-chloro-2-methylprop-1-enyl)-N-methyl aminomethylpolystyrene (1.55 mequiv/g, 6.96 g, 10.77 mequiv) in anhydrous acetonitrile (20 mL) was stirred at ambient temperature. Over 15 minutes, all of the BOCNH-lm-COOH dissolved and was converted to the corresponding acid chloride.

The reaction mixture was then filtered under a nitrogen atmosphere and the resin washed with 20 mL of dry acetonitrile. The acetonitrile filtrates containing the acid chloride were combined and added dropwise to a vigorously stirred 2-phase mixture of a solution of 4-amino-1-methyl-1H-imidazole-2-carboxylic acid methyl ester (643 mg, 4.15 mmol) in CH₂Cl₂ (20 mL) and a solution of Na₂CO₃ (572 mg, 5.40 mmol) in H₂O (20 mL). The resulting reaction mixture was stirred for 5 minutes, and then was diluted with CH₂Cl₂ and H₂O. The CH₂Cl₂ solution was isolated, dried (MgSO₄), and concentrated to afford a quantitative yield (1.64 g) of 4-[(4-tert-butoxycarbonylamino-1-methyl-1H-imidazole-2-carbonyl)-amino]-1-methyl-1H-imidazole-2-carboxylic acid methyl ester as a very pure off-white solid. Reverse phase HPLC of this material was carried out on an Agilent 1100 system using a Vydac 4.6×250 mm Protein & Peptide C18 column eluted at 1.2 mL/min with a linear gradient of 20% MeCN: 80% H₂O to 40% MeCN: 60% H₂O over a 15 minute period. Both solvents contained 0.1% TFA. A single 304 nm HPLC peak eluted at 12.223 minutes. ¹H NMR (CD₃CN): δ7.56 (s, 1H), 7.14 (br s, 1H), 4.01 (s, 3H), 4.00 (s, 3H), 3.90 (s, 3H), 1.51 (s, 9H).

### EXAMPLE 7

### Synthesis of 1-methyl-4-{[1-methyl-4-({1-methyl-4-[(1-methyl-1H-imidazole-2-carbonyl)-amino]-1H-imidazole-2-carbonyl}-amino)-1H-imidazole-2-carbonyl]-amino}-1H-pyrrole-2-carboxylic acid methyl ester using N-(1-chloro-2-methylprop-1-enyl)-N-methyl aminomethylpolystyrene

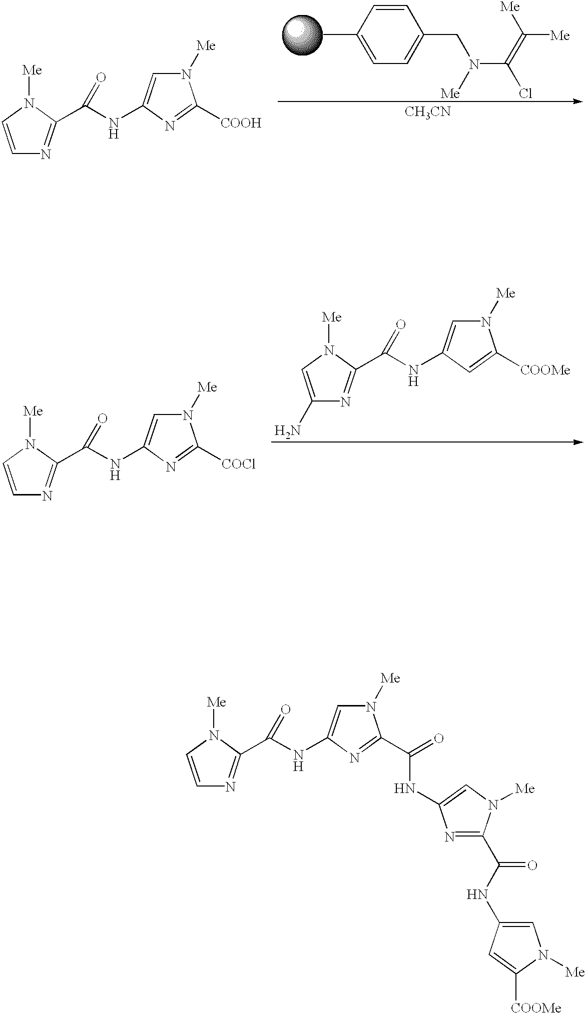

Trimethylsilyl triflate (150 μL, 0.81 mmol) was added in a single portion to a mixture of 1-methyl-4-[(1-methyl-1H-imidazole-2-carbonyl)-amino]-1H-imidazole-2-carboxylic acid (200 mg, 0.80 mmol) in anhydrous acetonitrile (8.00 mL). The solution which formed within a few moments was transferred to solid N-(1-chloro-2-methylprop-1-enyl)-N-methyl aminomethylpolystyrene (1.55 mequiv/g, 1.04 g, 1.61 mequiv). After stirring at ambient temperature for 10 minutes, an equal volume of anhydrous CHCl₃ (stabilized with amylenes) was added and the mixture was filtered in an inert atmosphere. The resin was washed with an additional 10 mL of anhydrous CHCl₃ (stabilized with amylenes), and the combined filtrates containing the 1-methyl-4-[(1-methyl-1H-imidazole-2-carbonyl)-amino]-1H-imidazole-2-carbonyl chloride were added dropwise to a vigorously stirred 2-phase mixture of a solution of 4-[(4-amino-1-methyl-1H-imidazole-2-carbonyl)-amino]-1-methyl-1H-pyrrole-2-carboxylic acid methyl ester (171 mg, 0.62 mmol) in CHCl₃ (10 mL), and a solution of Na₂CO₃ (170 mg, 1.60 mmol) in H₂O (5 mL). The resulting reaction mixture was stirred for 5 minutes, and then was diluted with CHCl₃ and H2O. The CHCl₃ solution was isolated, dried (MgSO₄), and concentrated to afford a 59% yield (240 mg) of 1-methyl-4-{[1-methyl-4-({1-methyl-4-[(1-methyl-1H-imidazole-2-carbonyl)-amino]-1H-imidazole-2-carbonyl}-amino)-1H-imidazole-2-carbonyl]-amino}-1H-pyrrole-2-carboxylic acid methyl ester as a very pure off-white solid. Reverse phase HPLC of this material was carried out on an Agilent 1100 system using a Vydac 4.6×250 mm Protein & Peptide C18 column eluted at 1.2 mL/min with a linear gradient of 20% MeCN: 80% H₂O to 40% MeCN: 60% H₂O over a 15 minute period. Both solvents contained 0.1% TFA. A single 304 nm HPLC peak eluted at 10.847 minutes. ¹H NMR (CD₃CN plus TFA): δ10.90 (s, 1H), 10.71 (s, 1H), 9.49 (s, 1H), 7.85 (s, 1H), 7.76 (s, 1H), 7.65 (m, 2H), 7.46 (d, J=1.8 Hz, 1H), 6.92 (d, J=1.9 Hz, 1H), 4.15 (s, 3H), 4.12 (s, 3H), 4.11 (s, 3H), 3.92 (s, 3H), 3.80 (s, 3H).

What is claimed is:

1. An amino acid halide comprising a 5-member heteroaromatic ring, the amino acid halide having a formula (4), (5) or (6): 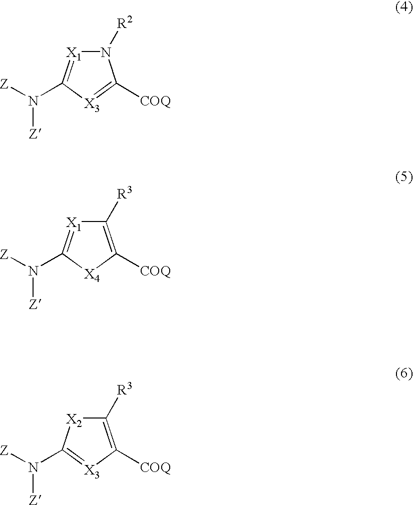
wherein:
Q is Cl or Br;
Z and Z′ are independently hydrogen or an amine protecting group which may be the same or different, provided that at least one of Z and Z′, together with the nitrogen atom to which they are attached, form a carbamate moiety;
X₁ is N or CR¹;
X₂ is O, S or NR¹;
X₃ is N or CR⁴;
X₄ is O or S;
R¹ is independently selected from hydrogen and substituted or unsubstituted alkyl;
R² is independently selected from hydrogen, substituted or unsubstituted C₁-C₁₀ alkyl, substituted or unsubstituted C₂-C₁₀ alkenyl, or substituted or unsubstituted C₂-C₁₀ alkynyl, provided that when X₃ is CH and X₁ is N, R² is not methyl;
R³ is independently selected from hydrogen, substituted or unsubstituted C₁-C₁₀ alkyl, substituted or unsubstituted C₂-C₁₀ alkenyl, substituted or unsubstituted C₂-C₁₀ alkynyl or halo, provided that (i) when X₂ is S and X₃ is N, R³ is not hydrogen, and (ii) when X₁ is N and X₄ is S, R³ is not hydrogen or methyl; and,
R⁴ is independently selected from hydrogen, hydroxy or alkoxy.

2. The amino acid halide of claim 1 having formula (5), wherein when X₄ is S and X₁ is N, R³ is substituted or unsubstituted C₂-C₁₀ alkyl.

3. The amino acid halide of claim 1 having formula (4), wherein when X₁ is N and X₃ is CH, R² is hydrogen or substituted or unsubstituted C₂-C₁₀ alkyl.

4. The amino acid halide of claim 1 having formula (6), wherein when X₃ is N and X₂ is S, R³ is substituted or unsubstituted C₁-C₁₀ alkyl.

5. The amino acid halide of claim 1 having the formula: 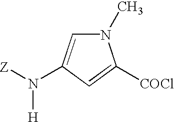
wherein Z is a protecting group as defined in claim 1.

6. The amino acid halide of claim 5 wherein Z is t-butoxycarbonyl.

7. The amino acid halide of claim 5 wherein Z is 9-fluoroenylmethoxycarbonyl.

8. The amino acid halide of claim 1 having the formula: 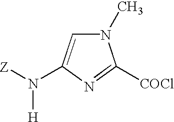
wherein Z is a protecting group as defined by claim 1.

9. The amino acid halide of claim 8 wherein Z is t-butoxycarbonyl.

10. The amino acid halide of claim 8 wherein Z is 9-fluoroenylmethoxycarbonyl.

11. The amino acid halide of claim 1 wherein the heteroaromatic ring is selected from pyrrole, imidazole, furan, pyrazole, thiophene, oxazole, thiazole and 1,2,4-triazole.

12. The amino acid halide of claim 1 having a formula (7A), (7B), (9A) or (9B): 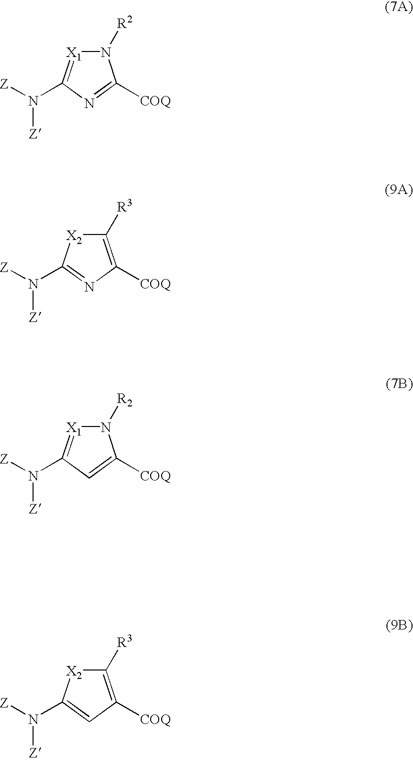
wherein: Q, Z, Z′, X₁, X₂, R¹, R² and R³ are as defined in claim 1.

13. The amino acid halide of claim 12 wherein Z is a protecting group and Z′ is hydrogen.

14. The amino acid halide of claim 13 wherein Z is t-butoxycarbonyl or 9-fluoroenylmethoxy carbonyl.

15. The amino acid halide of claim 14 wherein Q is Cl.

16. The amino acid halide of claim 15 having formula (7A) or (7B).

17. The amino acid halide of claim 16 wherein X₁ is CH and R² is H or CH₃.

18. The amino acid halide of claim 15 having formula (9A) or (9B).

19. The amino acid halide of claim 18 wherein X₂ is NH or NCH₃ and R³ is H or CH₃.

20. The amino acid halide of claim 18 wherein X₂ is O.

21. An amino acid halide oligomer comprising a 5-member heteroaromatic or a 6-member aromatic or heteroaromatic ring, the oligomer having a formula (10), (11) or (12): 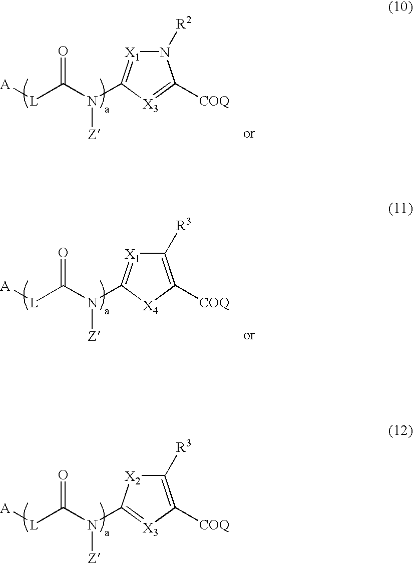
wherein:
Q is Cl or Br;
a is at least about 1 and represents the number of tandem units present in the oligomer;
A is H or NZZ′;
Z and Z′ are independently hydrogen or an amine protecting group which is the same or different, provided that at least one of Z and Z′, together with the nitrogen atom to which they are attached, form a carbamate moiety;
X₁ is N or CR¹;
X₂ is O, S, NR¹, —CR¹═CR¹′—, —CR¹═N—, —N═CR¹— or —N═N—;
X₃ is N or CR⁴;
X₄ is O or S;
R¹ and R¹′ are independently selected from hydrogen and substituted or unsubstituted alkyl;
R² is independently selected from hydrogen, substituted or unsubstituted C₁-C₁₀ alkyl, substituted or unsubstituted C₂-C₁₀ alkenyl, or substituted or unsubstituted C₂-C₁₀ alkynyl;
R³ is independently selected from hydrogen, substituted or unsubstituted C₁-C₁₀ alkyl, substituted or unsubstituted C₂-C₁₀ alkenyl, substituted or unsubstituted C₂-C₁₀ alkynyl or halo;
R⁴ is independently selected from hydrogen, hydroxy or alkoxy; and,
L is a subunit of the tandem unit independently selected from substituted or unsubstituted alkylene, substituted or unsubstituted arylene and substituted or unsubstituted heteroarylene, wherein (i) L may be the same or different for each tandem unit, and (ii) no more than about one L is present which enables a hairpin turn therein.

22. The oligomer of claim 21 wherein L is independently selected from substituted or unsubstituted ethylene, substituted or unsubstituted propylene, substituted or unsubstituted heteroarylene, and substituted or unsubstituted arylene.

23. The amino acid halide oligomer of claim 21 wherein a is an integer in the range of about 1 to about 10.

24. The amino acid halide oligomer of claim 23 wherein a is an integer in the range of about 2 to about 8.

25. The amino acid halide oligomer of claim 23 wherein a is an integer in the range of about 3 to about 5.

26. The amino acid halide oligomer of claim 23 having formula (11) wherein X₄ is O and X₁ is N or CH.

27. The amino acid halide oligomer of claim 23 having formula (11) wherein X₄ is S and X, is N or CH.

28. The amino acid halide oligomer of claim 23 having formula (12) wherein X₂ is NR¹ and X₃ is CH.

29. The amino acid halide oligomer of claim 23 having formula (10) wherein X₃ is CH and X₁ is N or CH.

30. The amino acid halide oligomer of claim 23 having formula (10) wherein X₃ is N and X₁ is N or CH.

31. The amino acid halide oligomer of claim 21 comprising a 5-member heteroaromatic ring and having a formula (16A), (16B), (18A) or (18B): 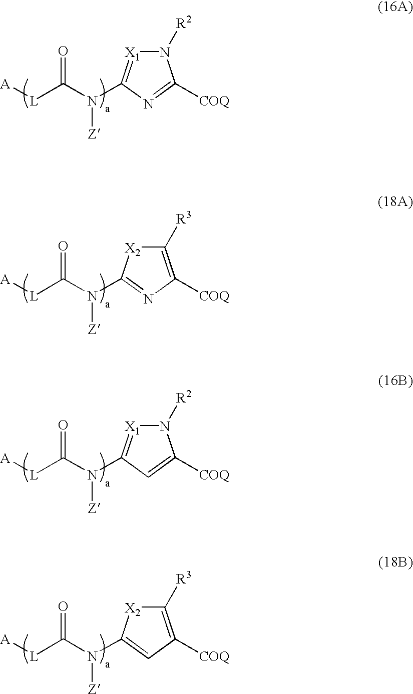
wherein: a, A, Q, Z, Z′, X₁, R¹, R², R³ and L are as defined in claim 23, and X₂ is O, S or NR¹.

32. The amino acid halide oligomer of claim 31 having formula (16A) or (16B).

33. The amino acid halide oligomer of claim 31 having formula (18A) or (18B).

34. The amino acid halide oligomer of claim 21 comprising a 6-membered aromatic or heteroaromatic ring.

35. The amino acid halide of claim 34 wherein the ring is selected from benzene, pyridine, pyrimidine, pyridazine, pyrazine and 1,2,4-triazine.

36. A process for preparing an amino acid halide, P—COQ, wherein P represents a substituted or unsubstituted, 5-member heteroaromatic or 6-member aromatic or heteroaromatic ring which has a protected amino group bound thereto, CO represents a carbonyl group bound to the ring, and Q represents a halo group bound to the carbonyl carbon, the process comprising contacting an immobilized α-haloenamine and a carboxylic acid, P—CO₂H, that corresponds structurally to the amino acid halide except that Q is hydroxy instead of halo.

37. The process of claim 36 wherein the amino acid halide has a formula (1), (2) or (3): 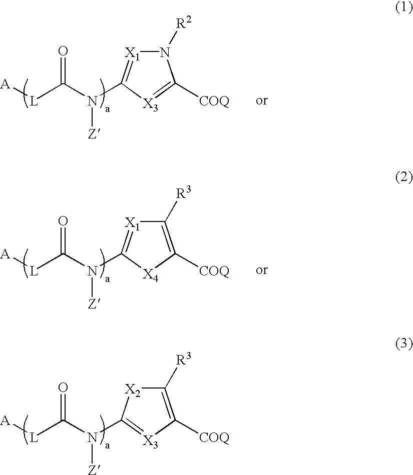
wherein:
Q is Cl or Br;
a is greater than or equal to 0 and, when greater than 0, represents the number of tandem units present in an oligomeric amino acid halide;
A is H or NZZ′, provided that when a is 0, A is NZZ′;
Z and Z′ are independently hydrogen or an amine protecting group which is the same or different, provided that at least one of Z and Z′, together with the nitrogen atom to which they are attached, form a carbamate moiety;
X₁ is N or CR¹;
X₂ isO,SorNR¹;
X₃ is N or CR⁴;
X₄ is O or S;
R¹ is independently selected from hydrogen and substituted or unsubstituted alkyl;
R² is independently selected from hydrogen, substituted or unsubstituted C₁-C₁₀ alkyl, substituted or unsubstituted C₂-C₁₀ alkenyl, or substituted or unsubstituted C₂-C₁₀ alkynyl;
R³ is independently selected from hydrogen, substituted or unsubstituted C₁-C₁₀ alkyl, substituted or unsubstituted C₂-C₁₀ alkenyl, substituted or unsubstituted C₂-C₁₀ alkynyl or halo;
R⁴ is independently selected from hydrogen, hydroxy or alkoxy; and,
L is a subunit of the tandem unit independently selected from substituted or unsubstituted alkylene, arylene and heteroarylene, wherein (i) L may be the same or different for each tandem unit present, and (ii) no more than about one L is present which enables a hairpin turn therein.

38. The process of claim 37 wherein the amino acid halide has the formula (1), and wherein X₃ is CH or N and X₁ is CH or N.

39. The process of claim 37 wherein the amino acid halide has the formula (3), and wherein X₂ is NH and X₃ is N or CH.

40. The process of claim 37 wherein the amino acid halide has the formula (2), wherein X₁ is CH or N and X₄ is O or S.

41. The process of claim 36 wherein the α-haloenamine has the formula: 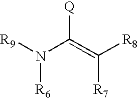
wherein:
R₆ and R₉ are independently hydrocarbyl, substituted hydrocarbyl, hydrocarbyloxy or substituted hydrocarbyloxy;
R₇ and R₈ are independently hydrogen, hydrocarbyl, substituted hydrocarbyl, hydrocarbylthio, substituted hydrocarbylthio, hydrocarbylcarbonyl, substituted hydrocarbylcarbonyl, hydrocarbyloxycarbonyl, substituted hydrocarbyloxycarbonyl, phosphinyl, thiophosphinyl, sulfinyl, sulfonyl, halo, cyano, or nitro, and
Q is chloro or bromo,
provided that at least one of R₆, R₇, R₈ and R₉ comprises a support which enables physical separation of the reagent from a liquid mixture.

42. The process of claim 36 wherein the resulting amino acid halide has a purity within a range of at least about 95 wt % to about 99.9 wt %.

43. The process of claim 36 wherein a yield of the resulting amino acid halide ranges from at least about 90% to about 99%.

44. The process of claim 36 wherein the amino acid halide and the α-haloenamine are contacted in a reaction zone for a duration of less than about 10 minutes.

45. A process for preparing an amino acid halide, the amino acid halide comprising a 5-member heteroaromatic or 6-member aromatic or heteroaromatic ring and having a formula (1), (2) or (3): 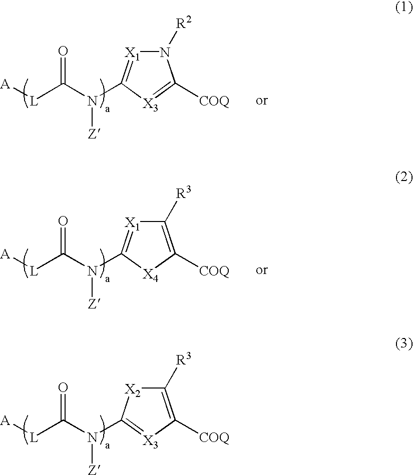
wherein:
Q is Cl or Br;
a is greater than or equal to 0 and, when greater than 0, represents the number of tandem units present in an oligomeric amino acid halide;
A is H or NZZ′, provided that when a is 0, A is NZZ′;
Z and Z′ are independently hydrogen or an amine protecting group which is the same or different, provided that at least one of Z and Z′, together with the nitrogen atom to which they are attached, form a carbamate moiety;
X₁ is N or CR¹;
X₂ is O, S, NR¹, —CR¹═CR¹′—, —CR¹═N—, —N═CR¹— or —N═N—;
X₃ is N or CR⁴;
X₄ is O or S;
R¹ and R¹′ are independently selected from hydrogen and substituted or unsubstituted alkyl;
R² is independently selected from hydrogen, substituted or unsubstituted C₁-C₁₀ alkyl, substituted or unsubstituted C₂-C₁₀ alkenyl, or substituted or unsubstituted C₂-C₁₀ alkynyl;
R³ is independently selected from hydrogen, substituted or unsubstituted C₁-C₁₀ alkyl, substituted or unsubstituted C₂-C₁₀ alkenyl, substituted or unsubstituted C₂-C₁₀ alkynyl or halo;
R⁴ is independently selected from hydrogen, hydroxy or alkoxy; and,
L is a subunit of the tandem unit independently selected from substituted or unsubstituted alkylene, arylene and heteroarylene, wherein (i) L may be the same or different for each tandem unit present, and (ii) no more than about one L is present which enables a hairpin turn therein; the process comprising contacting an α-haloenamine and an amino-protected, 5-member heteroaromatic or 6-member aromatic or heteroaromatic carboxylic acid, wherein said carboxylic acid corresponds to formula (1), (2) or (3) except that Q is OH and not Cl or Br.

46. The process of claim 45 wherein the resulting amino acid halide has a purity within a range of at least about 95 wt % to about 99.9 wt %.

47. The process of claim 45 wherein a yield of the resulting amino acid halide ranges from at least about 90% to about 99%.

48. The process of claim 45 wherein the amino acid halide and the α-haloenamine are contacted in a reaction zone for a duration of less than about 10 minutes.

49. The process of claim 45 wherein a is 0, the amino acid halide comprising a 5-member heteroaromatic ring and having a formula (4), (5) or (6): 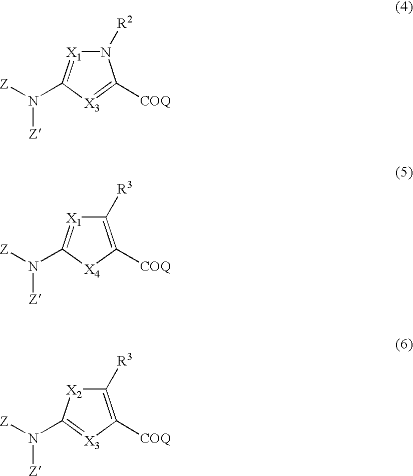
wherein:
Q, Z, Z′, X₁, X₃, X₄, R¹ and R⁴ are as defined in 45;
X₂ is O, S or NR¹;
R² is independently selected from hydrogen, substituted or unsubstituted C₁-C₁₀ alkyl, substituted or unsubstituted C₂-C₁₀ alkenyl, or substituted or unsubstituted C₂-C₁₀ alkynyl; and,
R³ is independently selected from hydrogen, substituted or unsubstituted C₁-C₁₀ alkyl, substituted or unsubstituted C₂-C₁₀ alkenyl, substituted or unsubstituted C₂-C₁₀ alkynyl or halo.

50. The process of claim 45 wherein the α-haloenamine has the formula: 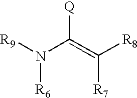
wherein:
R₆ and R₉ are independently hydrocarbyl, substituted hydrocarbyl, hydrocarbyloxy or substituted hydrocarbyloxy;
R₇ and R₈ are independently hydrogen, hydrocarbyl, substituted hydrocarbyl, hydrocarbylthio, substituted hydrocarbylthio, hydrocarbylcarbonyl, substituted hydrocarbylcarbonyl, hydrocarbyloxycarbonyl, substituted hydrocarbyloxycarbonyl, phosphinyl, thiophosphinyl, sulfinyl, sulfonyl, halo, cyano, or nitro, and
Q is chloro or bromo.

51. A process for preparing a polyamide oligomer or polymer, the process comprising:
(a) forming a population of amino-protected, amino acid halide building block units possessing a 5-member heteroaromatic or 6-member aromatic or heteroaromatic moiety therein, said units having a formula P—COQ, wherein P represents the aromatic or heteroaromatic moiety to which is bound a protected amino group, CO represents a carbonyl group bound to the aromatic or heteroaromatic moiety, and Q represents a halo group bound to the carbonyl carbon, the units being formed by contacting an α-haloenamine and a carboxylic acid, P—CO₂H, that corresponds structurally to the amino acid halide except that Q is hydroxy instead of halo;
(b) combining a member of the population of amino-protected, amino acid halide building block units with a moiety comprising an unprotected amino functionality to form a reaction product possessing an amide linkage and a protected amino functionality;
(c) deprotecting the amino group of the reaction product; and,
(d) repeating steps (b) and (c) at least once, wherein the deprotected reaction product formed in step (c) is used as the moiety comprising an unprotected amino functionality in a subsequent step (b).

52. The process of claim 51 wherein said acid halide has a purity within a range of at least about 95wt % to about 99.9 wt %.

53. The process of claim 51 wherein the moiety comprising an unprotected amino functionality is supported.

54. The process of claim 51 wherein the amino group nitrogen is protected as a carbamate.

55. The process of claim 51 wherein a ratio of equivalents of the amino-protected, amino acid halide building block units to the moiety comprising an unprotected amino functionality is within a range of about 1:1 to less than about 3:1.

56. The process of claim 51 wherein the yield of said reaction product is with a range of about 90% to about 99%.

57. The process of claim 51 wherein a member of the population of amino-protected, amino acid halide building block units is combined with a moiety comprising an unprotected amino functionality to form a reaction product for a duration of less than about 10 minutes.

58. The process of claim 51 wherein the α-haloenamine has the formula: 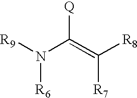
wherein:
R₆ and R₉ are independently hydrocarbyl, substituted hydrocarbyl, hydrocarbyloxy or substituted hydrocarbyloxy;
R₇ and R₈ are independently hydrogen, hydrocarbyl, substituted hydrocarbyl, hydrocarbylthio, substituted hydrocarbylthio, hyd rocarbylcarbonyl, substituted hydrocarbylcarbonyl, hydrocarbyloxycarbonyl, substituted hydrocarbyloxycarbonyl, phosphinyl, thiophosphinyl, sulfinyl, sulfonyl, halo, cyano, or nitro, and
Q is chloro or bromo,
provided that, when the α-haloenamine is immobilized, at least one of R₆, R₇, R₈ and R₉ comprises a support which enables physical separation of the reagent from a liquid mixture.

59. The process of claim 51 wherein the amino acid halide building block has a formula (1), (2) or (3): 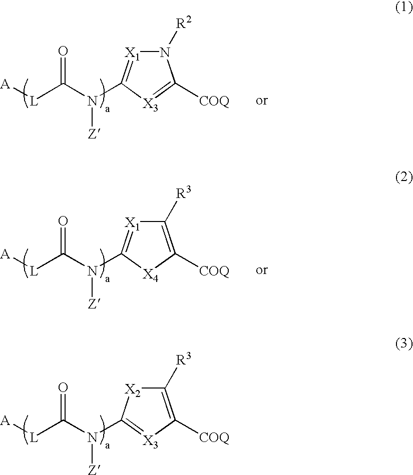
wherein:
Q is Cl or Br;
a is greater than or equal to 0 and, when greater than 0, represents the number of tandem units present in an oligomeric amino acid halide;
A is H or NZZ′, provided that when a is 0, A is NZZ′;
Z and Z′ are independently hydrogen or an amine protecting group which is the same or different, provided that at least one of Z and Z′, together with the nitrogen atom to which they are attached, form a carbamate moiety;
X₁ is N or CR¹;
X₂ is O, S or NR¹;
X₃ is N or CR⁴;
X₄ is O or S;
R¹ is independently selected from hydrogen and substituted or unsubstituted alkyl;
R² is independently selected from hydrogen, substituted or unsubstituted C₁-C₁₀ alkyl, substituted or unsubstituted C₂-C₁₀ alkenyl, or substituted or unsubstituted C₂-C₁₀ alkynyl;
R³ is independently selected from hydrogen, substituted or unsubstituted C₁-C₁₀ alkyl, substituted or unsubstituted C₂-C₁₀ alkenyl, substituted or unsubstituted C₂-C₁₀ alkynyl or halo;
R⁴ is independently selected from hydrogen, hydroxy or alkoxy; and,
L is a subunit of the tandem unit independently selected from substituted or unsubstituted alkylene, arylene and heteroarylene, wherein (i) L may be the same or different for each tandem unit present, and (ii) no more than about one L is present which enables a hairpin turn therein.

60. The process of claim 59 wherein a is 0.

61. The process of claim 60 wherein Q is Cl.

62. The process of claim 61 wherein Z is t-butoxycarbonyl.

63. The process of claim 61 wherein Z is 9-fluoroenylmethoxycarbonyl.

64. The process of claim 59 wherein a is an integer in the range of about 1 to about 10.

65. The process of claim 64 wherein a is an integer in the range of about 2 to about 8.

66. The process of claim 65 wherein Q is Cl.

67. The process of claim 66 wherein Z is t-butoxycarbonyl.

68. The process of claim 59 wherein steps (b), (c) and (d) are automated.

69. A process for preparing a polyamide oligomer or polymer, the process comprising:
(a) forming a population of amino-protected, amino acid halide building block units comprising a 5-member heteroaromatic ring and having a formula (4), (5) or (6): 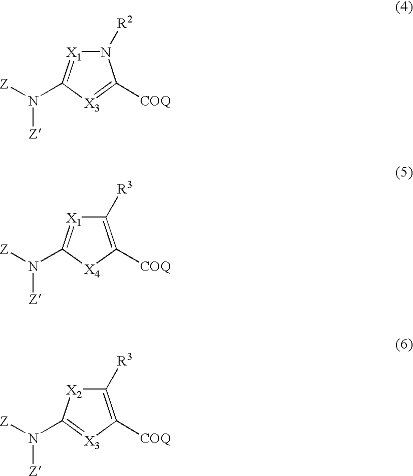
wherein:
Q is Cl or Br;
Z and Z′ are independently hydrogen or an amine protecting group which may be the same or different, provided that at least one of Z and Z′, together with the nitrogen atom to which they are attached, form a carbamate moiety;
X, is N or CR¹;
X₂ is O, S or NR¹;
X₃ is N or CR⁴;
X₄ is O or S;
R¹ is independently selected from hydrogen and substituted or unsubstituted alkyl;
R² is independently selected from hydrogen, substituted or unsubstituted C₁-C₁₀ alkyl, substituted or unsubstituted C₂-C₁₀ alkenyl, or substituted or unsubstituted C₂-C₁₀ alkynyl;
R³ is independently selected from hydrogen, substituted or unsubstituted C₁-C₁₀ alkyl, substituted or unsubstituted C₂-C₁₀ alkenyl, substituted or unsubstituted C₂-C₁₀ alkynyl or halo; and,
R⁴ is independently selected from hydrogen, hydroxy or alkoxy;
(b) combining a member of the population of amino-protected, amino acid halide building block units with a moiety comprising an unprotected amino functionality to form a reaction product possessing an amide linkage and a protected amino functionality;
(c) deprotecting the amino group of the reaction product; and, (d) repeating steps (b) and (c) at least once, wherein the deprotected reaction product formed in step (c) is used as the moiety comprising an unprotected amino functionality in a subsequent step (b).

70. The process of claim 69 wherein said amino acid halide building blocks have a formula (7A), (7B), (9A) or (9B): 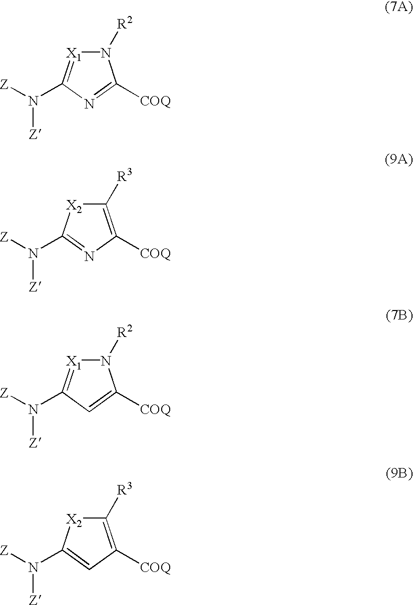
wherein: Q, Z, Z′, X₁, X₂, R¹, R² and R³ are as defined in 69.

71. The process of claim 69 wherein the amino acid halide has a purity within a range of at least about 95 wt % to about 99.9 wt %.

72. The process of claim 69 wherein a ratio of equivalents of the amino-protected, amino acid halide building block units to the moiety comprising an unprotected amino functionality is within a range of about 1:1 to less than about 3:1.

73. The process of claim 69 wherein steps (b), (c) and (d) are automated.

74. The process of claim 69 wherein the yield of said reaction product is with a range of about 90% to about 99%.

75. The process of claim 69 wherein a member of the population of amino-protected, amino acid halide building block units is combined with a moiety comprising an unprotected amino functionality to form a reaction product for a duration of less than about 10 minutes.
